(19) United States
(12) Patent Application Publication
(10) Pub. No.: US 2024/0229149 A1
(43) Pub. Date: Jul. 11, 2024

(54) 5-HYDROXYMETHYLATION ANALYSIS OF BUFFY COAT gDNA IN CANCER DETECTION

(71) Applicant: ClearNote Health, Inc., San Diego, CA (US)

(72) Inventors: Samuel LEVY, San Diego, CA (US); Gulfem Dilek GULER, San Diego, CA (US); Giulana P. MOGNOL, San Diego, CA (US); Yuhong NING, San Diego, CA (US)

(21) Appl. No.: 18/408,427

(22) Filed: Jan. 9, 2024

Related U.S. Application Data
(60) Provisional application No. 63/437,946, filed on Jan. 9, 2023.

(51) Int. Cl. C12Q 1/6886

(52) U.S. Cl. CPC C12Q 1/6886; C12Q 2600/154

(57) ABSTRACT

Methods for analyzing peripheral blood samples are provided, wherein buffy coat gDNA is analyzed to provide a buffy coat gDNA hydroxymethylation signature. The buffy coat gDNA hydroxymethylation signature is correlated with the presence of cancer in a patient. Combining the buffy coat gDNA hydroxymethylation signature with another feature value related to the patient increases the predictiveness of the process. In some embodiments, an additional feature value is the cell-free DNA (cfDNA) hydroxymethylation signature in a peripheral blood sample obtained from the same patient. The combination of feature values may be carried out using an ensemble-type analysis such as a stacked ensemble analysis.

### CROSS-REFERENCE TO RELATED APPLICATIONS

This application claims priority under 35 U.S.C. § 119(e)(1) to provisional U.S. patent application Ser. No. 63/437,946, filed Jan. 9, 2023. The disclosure of the aforementioned patent application is incorporated herein by reference in its entirety.

### TECHNICAL FIELD

The present invention relates generally to cancer, and more particularly relates to a novel hydroxymethylation analysis useful in an improved method for detecting cancer.

### BACKGROUND

Cancer is the second leading cause of death globally. Cancer mortality is exacerbated by diagnosis at late stage when prognosis is poor. Earlier cancer detection offers the opportunity to improve patient outcomes by identifying tumors when treatment is more likely to be effective. While breast, colorectal and lung cancers are among the few cancers for which screening modalities exist, screening tests that are currently used in the clinic can be expensive, invasive, and limited to detection of a single cancer type; this, in turn, may necessitate multiple tests, further increasing the cost for overall early cancer detection and resulting in possible delay of treatment. Liquid biopsy-based multi-cancer early detection tests aim to address these limitations and complement these screening approaches.

Current non-invasive methods for early cancer detection rely upon genetic, epigenetic, or proteomic changes in cell free DNA (cfDNA) that is obtained from plasma or in exosomes circulating in blood. While these methods can achieve a certain level of performance for cancer detection and therapy response prediction, there is an ongoing need to improve the performance of non-invasive tests to detect more cancers earlier (i.e., to increase sensitivity) and distinguish non-cancer cases from being identified falsely as positive cases (i.e., to increase specificity).

Peripheral blood contains multiple analytes that can be assessed and implemented in a non-invasive method for early detection of cancer. Among them, genomic and epigenomic profiling of plasma-derived cell free DNA (cfDNA) has been repeatedly shown to have utility for cancer detection (see, e.g., Liu et al. (2020), Ann. Oncol. 31: 745-759; Song et al. (2017) Cell Res 27: 1231-1242; Guler et al. (2020) Nat Commun. 11: 5270; Gao et al. (2022) Innovation 3: 100259). While cfDNA-based liquid biopsy assays are particularly effective with cancers having high levels of circulating tumor DNA (ctDNA), the sensitivity of these assays is reduced for early-stage disease when ctDNA levels are usually low (Gao et al. (2022), supra; Cohen et al. (2018) Science 359: 926-930; Chabon et al. (2020) Nature 580: 245-251). Therefore, the discovery of biomarkers that do not rely on ctDNA release is essential for improving early cancer detection. In addition to plasma, the buffy coat (BC) fraction of peripheral blood contains intact cells such as circulating tumor cells that have been widely investigated not only for cancer detection but also for cancer prognosis (Alix-Panabieres et al. (2016) Cancer Discov. 6: 479-491; Pascual et al. (2022) Ann. Oncol. 33: 750-768). Yet, the immune cells that make up the bulk of the buffy coat have been only minimally explored for their potential utility in cancer detection.

### SUMMARY OF THE INVENTION

The present invention provides a method for detecting cancer without a surgical biopsy or other invasive means, wherein the method can be carried out with respect to a wide range of cancer types and at various stages, including early stage cancer. The method is a “liquid biopsy” based technique that, in contrast to prior such methods, makes use of the buffy coat fraction of a patient's blood sample and a 5-hydroxymethylation analysis of the buffy coat. The method can be carried out without a combined analysis involving additional feature types, but, optimally, is combined with at least one additional feature. In some embodiments, the additional feature is a 5-hydroxymethylation signature obtained from a cell-free DNA (cfDNA) sample extracted from a blood sample of the same patient. The genes that are differentially hydroxymethylated in cancer in a buffy coat sample do not overlap significantly with genes that are differentially hydroxymethylated in a cfDNA sample, thereby enhancing the discriminatory power of a combined analysis.

In one embodiment, the invention provides a method for analyzing buffy coat in a peripheral blood sample obtained from a patient, wherein the method comprises:
obtaining a buffy coat hydroxymethylation signature for the patient by
extracting genomic DNA (gDNA) from the buffy coat without separating the buffy coat into individual cell types;
sequencing the gDNA in a manner that identifies 5-hydroxymethylcytosine (5hmC)-containing sites therein;
determining the extent of hydroxymethylation of the sequenced gDNA at each of a plurality of hydroxymethylation biomarker loci in a reference data set for a population group of individuals who have at least one shared characteristic, wherein the biomarker loci are preselected as differentially hydroxymethylated with respect to the at least one shared characteristic; and
reporting the extent of hydroxymethylation at each locus as the buffy coat hydroxymethylation signature.

As a primary application of the foregoing method is in the detection of cancer, the method may further comprise, in some embodiments, generating a buffy coat gDNA-based probability score that the patient has cancer from the buffy coat hydroxymethylation signature.

In some embodiments, the at least one shared characteristic comprises having cancer. In other embodiments, the at least one shared characteristic comprises not having cancer. In other embodiments, the at least one shared characteristic comprises having a particular type of cancer or not having a particular type of cancer. In additional embodiments, the at least one shared characteristic comprises having a particular stage of cancer or not having a particular stage of cancer.

In the aforementioned context, wherein the buffy coat hydroxymethylation analysis is implemented in a method for detecting cancer, each hydroxymethylation biomarker locus is selected as exhibiting differential hydroxymethylation in a manner that correlates with having cancer or a particular type of cancer.

In some embodiments, differential hydroxymethylation is determined using a p-value of less than or equal to 0.05 using a linear regression F-test.

In some embodiments, the method involves combining the buffy coat gDNA-based probability score alluded to above with at least one an additional feature value to characterize the likelihood that a patient has cancer.

In one embodiment, the additional feature comprises a cfDNA hydroxymethylation signature obtained from a cfDNA sample extracted from a blood sample taken from the same patient. In one aspect of the embodiment, the cfDNA sample is extracted from the same blood sample that comprises the buffy coat.

In other embodiments, the additional feature value derives from an additional feature type that comprises one or more of: DNA fragment size distribution; copy number variation; cfDNA concentration; methylation profile; T-cell-inflamed gene expression profile; circulating tumor DNA count; serum CA19-9 level; serum CA125 level; IDO-1 expression; T-cell count; T-cell percentage; inflammation gene signature; myeloid-derived suppressor cell count; lymphocyte count; deficient mismatch repair; tumor mutational burden; presence or absence of germline mutations; and a patient-specific clinical parameter.

The invention additionally provides a method for analyzing a peripheral blood sample obtained from a patient, wherein the method comprises:
(a) obtaining a buffy coat hydroxymethylation signature for the patient by
   (i) extracting genomic DNA (gDNA) from the buffy coat;
   (ii) sequencing the gDNA in a manner that identifies 5-hydroxymethylcytosine (5hmC)-containing sites therein;
   (iii) determining the extent of hydroxymethylation of the sequenced gDNA at each of a plurality of hydroxymethylation biomarker loci in a reference data set for a population group of individuals who have at least one shared characteristic, wherein the biomarker loci are preselected as differentially hydroxymethylated with respect to the at least one shared characteristic, thereby providing the buffy coat hydroxymethylation signature as the extent of hydroxymethylation at each locus;
(b) obtaining a cfDNA hydroxymethylation signature for the patient by
   (i) isolating cfDNA from plasma in a peripheral blood sample obtained from the patient;
   (ii) enriching for hydroxymethylated DNA in the cfDNA, amplifying the hydroxymethylated DNA, sequencing the amplified hydroxymethylated DNA in a manner that identifies 5-hydroxymethylcytosine (5hmC)-containing fragments in the DNA, and determining the extent of hydroxymethylation of the sequenced cfDNA at each of a plurality of cfDNA hydroxymethylation biomarker loci preselected as differentially hydroxymethylated with respect to the at least one shared characteristic, thereby providing the cfDNA hydroxymethylation signature as the extent of hydroxymethylation at each locus; and
(c) combining the buffy coat gDNA hydroxymethylation signature and the cfDNA hydroxymethylation signature in a manner that provides a classification model suitable for generating a composite probability score representing the likelihood that the patient has cancer or a particular type of cancer.

In some embodiments, the composite probability score represents the likelihood that the patient has cancer.

In some embodiments, the composite probability score represents the likelihood that the patient has a particular type of cancer. In certain aspects of these embodiments, the particular type of cancer is breast cancer. In other aspects, the type of cancer is colorectal cancer. In still other aspects, the type of cancer is lung cancer.

The aforementioned method may further include combining the composite probability score with an additional feature value for at least one additional feature type to characterize the likelihood that the patient has cancer. In some embodiments, the additional feature value derives from an additional feature type that comprises one or more of: DNA fragment size distribution; copy number variation; cfDNA concentration; methylation profile; T-cell-inflamed gene expression profile; circulating tumor DNA count; serum CA19-9 level; serum CA125 level; IDO-1 expression; T-cell count; T-cell percentage; inflammation gene signature; myeloid-derived suppressor cell count; lymphocyte count; deficient mismatch repair; tumor mutational burden; presence or absence of germline mutations; and a patient-specific clinical parameter. In some embodiments, the additional feature type comprises: the number of cfDNA fragments in each of at least two nonoverlapping size ranges; copy number variation in the cfDNA sample; concentration of cfDNA in the cfDNA sample; a patient-specific clinical parameter; and combinations of any of the foregoing. Representative patient-specific clinical parameters include, without limitation, lesion size; lesion grade; lesion stage; lesion location; patient age; patient weight; patient gender; patient ethnicity; cigarette smoking status; and exposure or lack of exposure to a known carcinogen.

In some embodiments, combining two or more feature values comprises an ensemble analysis. In some embodiments, combining two or more feature values comprises a stacked ensemble analysis. In one aspect of the aforementioned embodiments, the buffy coat hydroxymethylation signature is used as a base model in a stacked ensemble analysis.

The invention additionally provides a method for analyzing a peripheral blood sample obtained from a patient which comprises:
(a) isolating a specific cell type from the buffy coat layer of a peripheral blood sample obtained from the patient;
(b) obtaining a buffy coat cell-specific hydroxymethylation signature for the patient by
   (i) extracting genomic DNA (gDNA) from the isolated specific cell type;
   (ii) sequencing the gDNA in a manner that identifies 5-hydroxymethylcytosine (5hmC)-containing sites therein; and
   (iii) determining the extent of hydroxymethylation of the sequenced gDNA at each of a plurality of hydroxymethylation biomarker loci in a reference data set for a population group of individuals who have at least one shared characteristic, wherein the biomarker loci are preselected as differentially hydroxymethylated with respect to the at least one shared characteristic, thereby providing the buffy coat cell-specific hydroxymethylation signature as the extent of hydroxymethylation at each locus;
(c) isolating cfDNA from plasma in a peripheral blood sample obtained from the same patient;
(d) obtaining a cfDNA hydroxymethylation signature for the patient by enriching for hydroxymethylated DNA in the cfDNA, amplifying the hydroxymethylated DNA, sequencing the amplified hydroxymethylated DNA in a manner that identifies 5-hydroxymethylcytosine (5hmC)-containing fragments in the DNA, and determining the extent of hydroxymethylation of the sequenced cfDNA at each of a plurality of cfDNA hydroxymethylation biomarker loci preselected as differentially hydroxymethylated with respect to the at least one shared characteristic, thereby providing the cfDNA hydroxymethylation signature as the extent of hydroxymethylation at each locus; and
(e) combining the buffy coat cell-specific hydroxymethylation signature and the cfDNA hydroxymethylation signature in a manner that provides a classification model suitable for generating a composite probability score representing the likelihood that the patient has cancer or a particular type of cancer.

In some embodiments, the specific cell type is a granulocyte. In a related embodiment, the invention provides a method for detecting cancer in a patient by determining the percentage of granulocytes in a buffy coat fraction obtained from a peripheral blood sample and comparing that percentage to an established percentage of granulocytes in a reference standard, wherein the reference standard may be a mean percentage observed in non-cancer patients or a mean percentage observed in cancer patients. An elevated percentage of granulocytes in the buffy coat has now been found to correlate with the likelihood that a patient has cancer. Granulocyte percentage is itself correlated with the presence of cancer, but may also be combined as a feature type with buffy coat hydroxymethylation signature and/or cfDNA hydroxymethylation signature.

### BRIEF DESCRIPTION OF THE DRAWINGS

The file of this patent contains at least one drawing executed in color. Copies of this patent with color drawings will be provided by the Patent and Trademark Office upon request and payment of the necessary fee.

FIG. 1 is a table summarizing the clinical characteristics of the cancer and non-cancer cohorts evaluated in the Example, showing the distribution of age, sex, and smoking status in each cohort. Also shown is an indication of the composition of the cancer cohort, stratified by individual tumor types, and the stage distribution within the cancer cohort.

FIG. 2 schematically illustrates the laboratory process and analytical steps employed in one embodiment of the invention, and as described in the Example. Plasma or buffy coat were isolated from whole blood obtained by routine venous phlebotomy. cfDNA and gDNA were then extracted from plasma and BC, respectively. WGS and 5hmC libraries, prepared from both cfDNA and fragmented gDNA, were then sequenced. Cancer prediction models were built using sequencing data obtained from cfDNA and BC individually or in combination.

FIGS. 3-8 pertain to differential 5hmC features in cancer versus non-cancer samples from the BC gDNA and performance of the BC cancer prediction model, as described in the Example herein:

FIG. 3 is an MA plot indicating differentially hydroxymethylated genes (DhMGs) identified in cancer samples compared to non-cancer controls. Red and blue dots respectively indicate increased or decreased 5hmC density in cancer relative to non-cancers with a false discovery rate (FDR)≤0.05.

FIG. 4 provides boxplots (log CPM) of selected (FDR<0.001) DhMGs in all cancer samples versus non-cancers for purposes of comparison. The center line represents the median and the bounds of the box represent 5th through 95th percentiles. Each dot represents an individual gDNA sample.

FIG. 5 indicates the relative change in the percentage of granulocytes, monocytes and lymphocytes as determined by flow cytometry. The final percentage of cells was determined by multiplying the percentage of CD45⁺ cells by the percentage of CD14⁻CD15⁺(granulocytes), CD14⁺CD15⁻ (monocytes), or double-negative CD15⁻CD14⁻ cells (lymphocytes). The plots indicate an increase relative to the non-cancer cells (n=148 cancers and n=165 non-cancers) and * indicates statistical significance between early-stage and late-stage cancers (F-test, p<0.001) compared to the non-cancer samples.

FIG. 6 provides gene set enrichment analysis (GSEA) C8 normalized enrichment scores of the top hematopoietic-related positive and negative representative pathways in cancer and non-cancer samples.

FIG. 7 is a cross-validation ROC curve showing the performance of the BC model to distinguish all cancer samples relative to non-cancer controls. AUC value with confidence interval [CI] are shown. The red dashed line represents 98% specificity.

FIG. 8 indicates in graph form the cancer prediction scores obtained using the BC model stratified by cancer type. The number of true positives, for the cancer cohorts, and false positives, for non-cancer controls, over the total number of samples are indicated underneath the graph.

FIGS. 9-11 relate to the observation and analysis of 5hmC signals present in cfDNA in cancer and non-cancer samples and the performance of the cfDNA cancer prediction model, as described in the Example herein:

FIG. 9 is an MA plot indicating DhMGs in cfDNA, comparing all cancer versus non-cancer samples (FDR≤0.05). The red and blue dots indicate increased or decreased 5hmC density in cancer compared to non-cancer samples, respectively.

FIG. 10 is a cross-validation ROC curve showing the performance of the cfDNA model in distinguishing all cancer versus non-cancer samples, with AUC value and confidence interval [CI] shown. The red dashed line represents 98% specificity.

FIG. 11 indicates in graph form the cancer prediction scores obtained using the cfDNA model stratified by cancer type. As in FIG. 8, the number of true positives, for the cancer cohorts, and false positives, for non-cancer controls, over the total number of samples are indicated underneath the graph.

FIG. 12 are Venn diagrams showing the overlap of differentially hydroxymethylated genes in breast cancer, colorectal cancer, and lung cancer relative to non-cancer controls.

FIG. 13 provides gene set enrichment analysis (GSEA) C8 normalized enrichment scores of the top hematopoietic-related positive and negative representative pathways in cancer and non-cancer samples.

FIGS. 14-17 relate to the comparison of cancer hydroxymethylome changes observed between the BC and matched cfDNA samples, as described in the Example herein:

FIG. 14 is a Venn diagram of DhMGs identified by comparing 152 cancer samples to 166 non-cancer samples in BC and in cfDNA (Fisher's exact test, p-value=1).

FIG. 15 indicates the correlation of cancer to non-cancer fold change in 5hmC counts across all genes as calculated using the BC and cfDNA models (scatter plot of the two datasets); p-value (linear regression F-test)<0.001.

FIG. 16 is an MA plot of DhMGs observed using the BC model, comparing all cancers versus non-cancers samples (FDR≤0.05). Red and blue dots indicate increased or decreased 5hmC density in cancer compared to non-cancers, respectively. n=71 early-stage cancers (breast n=35, colorectal n=19, lung n=17) and 166 non-cancers.

FIG. 17 is an analogous MA plot of DhMGs observed using the cfDNA model.

FIGS. 18-23 pertain to the performance of the BC-cfDNA combined cancer prediction model as described in the Example:

FIG. 18 is a cross-validation ROC curve showing the performance of the combined model to distinguish all cancer versus non-cancer samples, with AUC value and confidence interval [CI] shown. The red dashed line represents 98% specificity.

FIG. 19 provides cancer prediction scores stratified by cancer type using the combined BC and cfDNA model, indicating the number of true positives, for the cancer cohorts, and false positives, for non-cancer controls, underneath the graph.

FIG. 20 is a Venn diagram showing the overlap of true positives scored using the BC, cfDNA or the combined models, set at 98% training specificity.

FIG. 21 is a Venn diagram showing the overlap of false positives scored using the BC, cfDNA or the combined models, set at 98% training specificity.

FIGS. 22 and 23 provide a cross-validation performance comparison among the BC, cfDNA and the combined models, at 98% training specificity for: all cancers or individual cancer types versus non-cancer samples (FIG. 22); and all early-stage cancer (stages I-II) versus non-cancers (FIG. 23). * indicates statistical significance (p<0.05, McNemar's test) between the individual models relative to the combined model.

FIG. 24 is a dot plot providing a comparison between granulocyte percentage and BC prediction scores, as evaluated in the Example. The percentage of granulocytes (CD45⁺CD14⁻CD15⁺ cells) in the BC was determined by flow cytometry. This result was plotted against each sample's prediction score determined by the BC model.

FIGS. 25-27 pertain to differential 5hmC features identified in granulocytes and monocytes of cancer versus non-cancer BC samples and performance of their cancer prediction models, as described in the Example herein:

FIG. 25 is a table summarizing the clinical cohort characteristics in the granulocyte cohort.

FIG. 26 is a table summarizing the clinical cohort characteristics in the monocyte cohort.

FIG. 27 provides MA plots of DhMG observed in BC for granulocytes (left plot) and monocytes (right plot), comparing all cancer and non-cancer samples (FDR≤0.05). Red and blue dots indicate increased or decreased 5hmC density in cancer compared to non-cancer samples, respectively.

FIG. 28 provides cross-validation ROC curves showing the performance of the granulocyte (left curve) and monocyte (right curve) models to distinguish cancer from non-cancer samples, with AUC value and confidence interval [CI] shown.

FIG. 29 is a table setting forth identifying genes with differential 5hmC in buffy coat-derived gDNA obtained from individuals with cancer compared to non-cancer controls as determined by thresholding with FDR≤0.05 and | fold change|>1.25).

FIG. 30 is a table indicating GSEA results with cell type signature (C8) gene sets comparing cancers to non-cancer using 5hmC counts over genes in buffy coat-derived gDNA.

FIG. 31 is a table showing performance of the cancer prediction model built by buffy coat features alone, cfDNA features alone or a combination of feature sets from buffy coat and the matched cfDNA.

FIG. 32 is a table indicating those genes with differential 5hmC in cfDNA obtained from individuals with cancer compared to non-cancer controls (FDR≤0.05 and | fold change|>1.1).

FIG. 33 provides a comparison of sensitivity values at 98% specificity for cancer prediction models that were built with buffy coat features alone, cfDNA features alone or combination of buffy coat and cfDNA features.

### DETAILED DESCRIPTION OF THE INVENTION

Unless defined otherwise, all technical and scientific terms used herein have the meaning commonly understood by one of ordinary skill in the art to which the invention pertains. Specific terminology of particular importance to the description of the present invention is defined below. Other relevant terminology is defined in International Patent Publication No. WO 2017/176630 to Quake et al. for “Noninvasive Diagnostics by Sequencing 5-Hydroxymethylated Cell-Free DNA.” The aforementioned patent publication as well as all other patent documents and publications referred to herein are expressly incorporated by reference.

In this specification and the appended claims, the singular forms “a,” “an” and “the” include plural referents unless the context clearly dictates otherwise.

Numeric ranges are inclusive of the numbers defining the range. Unless otherwise indicated, nucleic acids are written left to right in 5′ to 3′ orientation; amino acid sequences are written left to right in amino to carboxy orientation, respectively.

The headings provided herein are not limitations of the various aspects or embodiments of the invention. Accordingly, the terms defined immediately below are more fully defined by reference to the specification as a whole.

The term “sample” as used herein relates to a material or mixture of materials, typically in liquid form, containing one or more analytes of interest. The biological samples evaluated herein are blood samples obtained from a patient.

A “nucleic acid sample” as that term is used herein refers to a biological sample comprising nucleic acids. The nucleic acid sample may be a genomic DNA sample, or it may be comprised of cell-free DNA wherein the sample is substantially free of histones and other proteins, such as will be the case following cell-free DNA purification.

A “sample fraction” refers to a subset of an original biological sample, and may be a compositionally identical portion of the biological sample, as when a blood sample is divided into identical fractions. Alternatively, the sample fraction may be compositionally different, as will be the case when, for example, certain components of the biological sample are removed, with extraction of cell-free nucleic acids being one such example.

As used herein, the term “cell-free nucleic acid” encompasses both cell-free DNA and cell-free RNA, where the cell-free DNA and cell-free RNA may be in a cell-free fraction of a biological sample comprising a body fluid. The body fluid may be blood, including peripheral blood, serum, or plasma. In most instances, the biological sample is a blood sample, and a cell-free nucleic acid sample, e.g., a cell-free DNA sample, is extracted therefrom using now-conventional means known to those of ordinary skill in the art and/or described in the pertinent texts and literature; kits for carrying out cell-free nucleic acid extraction are commercially available (e.g., the AllPrep® DNA/RNA Mini Kit and QIAmp DNA Blood Mini Kit, both available from Qiagen, or the MagMAX Cell-Free Total Nucleic Acid Kit and the MagMAX DNA Isolation Kit, available from ThermoFisher Scientific). Also see, e.g., Hui et al. Fong et al. (2009) Clin. Chem. 55(3):587-598.

“Adapters” as that term is used herein are short synthetic oligonucleotides that serve a specific purpose in a biological analysis. Adapters can be single-stranded or double-stranded, although the preferred adapters herein are double-stranded. In one embodiment, an adapter may be a hairpin adapter (i.e., one molecule that base pairs with itself to form a structure that has a double-stranded stem and a loop, where the 3′ and 5′ ends of the molecule ligate to the 5′ and 3′ ends of a double-stranded DNA molecule, respectively). In another embodiment, an adapter may be a Y-adapter. In another embodiment, an adapter may itself be composed of two distinct oligonucleotide molecules that are base paired with each other. As would be apparent, a ligatable end of an adapter may be designed to be compatible with overhangs made by cleavage by a restriction enzyme, or it may have blunt ends or a 5′ T overhang. The term “adapter” refers to double-stranded as well as single-stranded molecules. An adapter can be DNA or RNA, or a mixture of the two. An adapter containing RNA may be cleavable by RNase treatment or by alkaline hydrolysis. An adapter may be 15 to 100 bases, e.g., 50 to 70 bases, although adapters outside of this range are envisioned.

The term “adapter-ligated,” as used herein, refers to a nucleic acid that has been ligated to an adapter. The adapter can be ligated to a 5′ end and/or a 3′ end of a nucleic acid molecule. As used herein, the term “adding adapter sequences” refers to the act of adding an adapter sequence to the end of fragments in a sample. This may be done by filling in the ends of the fragments using a polymerase, adding an A tail, and then ligating an adapter comprising a T overhang onto the A-tailed fragments. Adapters are usually ligated to a DNA duplex using a ligase, while with RNA, adapters are covalently or otherwise attached to at least one end of a cDNA duplex preferably in the absence of a ligase.

The term “amplifying” as used herein refers to generating one or more copies, or “amplicons,” of a template nucleic acid, such as may be carried out using any suitable nucleic acid amplification technique, such as technology, such as PCR, NASBA, TMA, and SDA.

The terms “enrich” and “enrichment” refer to a partial purification of template molecules that have a certain feature (e.g., nucleic acids that contain 5-hydroxymethylcytosine) from analytes that do not have the feature (e.g., nucleic acids that do not contain hydroxymethylcytosine). Enrichment typically increases the concentration of the analytes that have the feature by at least 2-fold, at least 5-fold or at least 10-fold relative to the analytes that do not have the feature. After enrichment, at least 10%, at least 20%, at least 50%, at least 80% or at least 90% of the analytes in a sample may have the feature used for enrichment. For example, at least 10%, at least 20%, at least 50%, at least 80% or at least 90% of the nucleic acid molecules in an enriched composition may contain a strand having one or more hydroxymethylcytosines that have been modified to contain a capture tag.

The term “sequencing,” as used herein, refers to a method by which the identity of at least 10 consecutive nucleotides (e.g., the identity of at least 20, at least 50, at least 100 or at least 200 or more consecutive nucleotides) of a polynucleotide is obtained.

The terms “next-generation sequencing” (NGS) or “high-throughput sequencing”, as used herein, refer to the so-called parallelized sequencing-by-synthesis or sequencing-by-ligation platforms currently employed by Illumina, Life Technologies, Roche, etc. Next-generation sequencing methods may also include nanopore sequencing methods such as that commercialized by Oxford Nanopore Technologies, electronic detection methods such as Ion Torrent technology commercialized by Life Technologies, and single-molecule fluorescence-based methods such as that commercialized by Pacific Biosciences.

The term “read” as used herein refers to the raw or processed output of sequencing systems, such as massively parallel sequencing. In some embodiments, the output of the methods described herein is reads. In some embodiments, these reads may need to be trimmed, filtered, and aligned, resulting in raw reads, trimmed reads, aligned reads.

A “Unique Feature Identifier” (UFI) sequence refers to a relatively short nucleic acid sequence that serves to identify a feature of a nucleic acid molecule. Nucleic acid template molecules and amplicons thereof that contain a UFI are sometimes referred to herein as “barcoded” template molecules or amplicons. Examples of UFI sequence types include, without limitation, the following:

A “molecular UFI sequence” (or “molecular barcode”) is appended to every nucleic acid template molecule in a sample, and is random, such that, providing the UFI sequence is of sufficient length, every nucleic acid template molecule is attached to a unique UFI sequence. Molecular UFI sequences, as is known in the art, can be used to account for and offset amplification and sequencer errors, allow a user to track duplicates and remove them from downstream analysis, and enable molecular counting, and, in turn, the determination of an analyte concentration. See, e.g., Casbon et al. (2011) Nuc. Acids Res. 39(12):1-8. The “unique feature” here is the identity of the nucleic acid template molecules.

In some embodiments, a UFI may have a length in the range of from 1 to about 35 nucleotides, e.g., from 3 to 30 nucleotides, 4 to 25 nucleotides, or 6 to 20 nucleotides. In certain cases, the UFI may be error-detecting and/or error-correcting, meaning that even if there is an error (e.g., if the sequence of the molecular barcode is mis-synthesized, mis-read or distorted during any of the various processing steps leading up to the determination of the molecular barcode sequence) then the code can still be interpreted correctly. The use of error-correcting sequences is described in the literature (e.g., in U.S. Patent Publication Nos. U.S. 2010/0323348 to Hamati et al. and U.S. 2009/0105959 to Braverman et al., both of which are incorporated herein by reference).

The term “detection” is used interchangeably with the terms “determining,” “measuring,” “evaluating,” “assessing,” “assaying,” and “analyzing,” to refer to any form of measurement, and include determining if an element is present or not. These terms include both quantitative and/or qualitative determinations. Assessing may be relative or absolute. “Assessing the presence of” thus includes determining the amount of a moiety present, as well as determining whether it is present or absent. Assessing the level at a hydroxymethylation biomarker locus refers to a determination of the degree of hydroxymethylation at that locus.

“Accuracy” refers to the degree of conformity of a measured or calculated quantity (a test reported value) to its accurate (or true) value. Clinical accuracy relates to the proportion of true outcomes (true positives (TP) or true negatives (TN) versus misclassified outcomes (false positives (FP) or false negatives (FN), and may be stated as a sensitivity, specificity, positive predictive values (PPV) or negative predictive values (NPV), or as a likelihood, or odds ratio, among other measures.

“Performance” is a term that relates to the overall usefulness and quality of a diagnostic or prognostic test, including, among others, clinical and analytical accuracy, other analytical and process characteristics, such as use characteristics (e.g., stability, ease of use), health economic value, and relative costs of components of the test. Any of these factors may be the source of superior performance and thus usefulness of the test, and may be measured by appropriate “performance metrics,” such as AUC, time to result, shelf life, etc. as relevant.

“Clinical parameters” encompass all non-sample biomarkers of subject health status or other characteristics, such as, without limitation, lesion size; lesion location; patient age; patient weight; patient gender; patient ethnicity; family history; genetic mutations; and PD-L1 tumor staining result, which is currently used in the clinic to determine whether anti-PD-1 therapy is in order.

A “formula,” “algorithm,” or “model” is any mathematical equation, algorithmic, analytical or programmed process, or statistical technique that takes one or more continuous or categorical inputs and calculates an output value, sometimes referred to as a “probability score” or “index value.” Non-limiting examples of “formulas” include sums, ratios, and regression operators, such as coefficients or exponents, biomarker value transformations and normalizations (including, without limitation, those normalization schemes based on clinical parameters, such as gender, age, or ethnicity), rules and guidelines, statistical classification models, and neural networks trained on historical populations.

Of particular use in combining hydroxymethylation levels at various biomarker loci and clinical parameters, optionally in further combination with other factors (e.g., non-hydroxymethylation biomarkers), are linear and non-linear equations and statistical classification analyses to determine the correlation between hydroxymethylation levels at the biomarker loci detected in a patient sample and the patient's likelihood of having a particular type of cancer. In panel and combination construction, of particular interest are structural and syntactic statistical classification algorithms, and methods of risk index construction, utilizing pattern recognition and machine learning features, including established techniques such as cross-correlation, Principal Components Analysis (PCA), factor rotation, Logistic Regression (Log Reg), Linear Discriminant Analysis (LDA), Eigengene Linear Discriminant Analysis (ELDA), Support Vector Machines (SVM), Random Forest (RF), Recursive Partitioning Tree (RPART), as well as other related decision tree classification techniques, Shrunken Centroids (SC), StepAIC, Kth-Nearest Neighbor, Boosting, Decision Trees, Neural Networks, Bayesian Networks, and Hidden Markov Models, among others. Many such algorithmic techniques have been further implemented to perform both feature (loci) selection and regularization, such as in ridge regression, lasso, and elastic net, among others. Other techniques may be used in survival and time to event hazard analysis, including Cox, Weibull, Kaplan-Meier and Greenwood models well known to those of skill in the art. Many of these techniques are useful either combined with a hydroxymethylation biomarker selection technique, such as forward selection, backwards selection, or stepwise selection, complete enumeration of all potential biomarker sets, or panels, of a given size, genetic algorithms, or they may themselves include biomarker selection methodologies. These may be coupled with information criteria, such as Akaike's Information Criterion (AIC) or Bayes Information Criterion (BIC), in order to quantify the tradeoff between additional biomarkers and model improvement, and to aid in minimizing overfit. The resulting predictive models may be validated in other studies, or cross-validated in the study they were originally trained in, using such techniques as Bootstrap, Leave-One-Out (LOO) and 10-Fold cross-validation (10-Fold CV). At various steps, false discovery rates may be estimated by value permutation according to techniques known in the art.

“Likelihood,” in the context of one embodiment of the present invention, is the probability that a patient has or does not have cancer or a particular type of cancer.

A “hydroxymethylation level” refers to the extent of hydroxymethylation within a hydroxymethylation biomarker locus. The extent of hydroxymethylation is normally measured as hydroxymethylation density, e.g., the ratio of 5hmC residues to total cytosines, both modified and unmodified, within a nucleic acid region. Other measures of hydroxymethylation density are also possible, e.g., the ratio of 5hmC residues to total nucleotides in a nucleic acid region.

A “hydroxymethylation profile” or “hydroxymethylation signature” refers to a data set that comprises the hydroxymethylation level at each of a plurality of hydroxymethylation biomarker loci that are preselected as differentially hydroxymethylated with regard to a particular disease phenotype, e.g., lung cancer, colorectal cancer, breast cancer, or the like. The hydroxymethylation profile may be a reference hydroxymethylation profile that comprises composite a hydroxymethylation profile for a population of individuals with at least one shared characteristic, as explained elsewhere herein. The hydroxymethylation profile may also be a patient hydroxymethylation signature, constructed from the measurement of hydroxymethylation levels at each of a plurality of hydroxymethylation biomarker sites.

The term “locus” as used throughout this application refers to a site on a nucleic acid molecule, wherein the nucleic acid molecule may be single-stranded or double-stranded, and further wherein an individual locus (or multiple “loci”) may be of any length, thus including a single CpG site as well as a full-length gene, or across larger features such as topologically associated domains, including when several such loci are aggregated into groups such as related sequence motifs, other homologies or functional characteristics (regardless of their adjacency or topological relationship). The loci herein may be contained within a gene body; within an annotation feature outside of the gene body, such as a promoter, an enhancer, a transcription initiation site, a transcription stop site, or a DNA binding site, or a combination thereof; or within an untranslated region, or “UTR” (including 3′UTRs and 5′UTRs).

It should be noted that some of the individual hydroxymethylation biomarkers disclosed herein may not have significant individual significance in a particular evaluation, but when used in combination with one or more other types of biomarkers and, optionally, clinical parameters impacting on the detection and evaluation of a cancerous lesion become significant in discriminating as a method of the invention requires.

For the purpose of this application, any two variables are considered to be “very highly correlated” when they have a Coefficient of Determination (R2) of 0.5 or greater. The present invention encompasses such functional and statistical equivalents to the presently disclosed hydroxymethylation biomarkers.

The term “correlate” as used herein in reference to two variables (e.g., two values, two sets of values, a value or value set and a disease state, a value or set of values and a risk associated with the disease state, or the like) indicates a tendency of the two variables to vary together. A “correlation” is a measure of the extent to which two or more variables fluctuate together. A positive correlation indicates the extent to which those variables increase or decrease in parallel. One example of a positive correlation is the relationship between a hydroxymethylation level at a hydroxymethylation biomarker locus, on the one hand, and the likelihood that a patient has cancer or a particular type of cancer, on the other. Conversely, a negative correlation would exist when the hydroxymethylation level at a hydroxymethylation biomarker locus decreases as a subject's likelihood of having cancer or a particular type of cancer decreases.

As explained elsewhere herein, the present invention relates, in part, to the discovery that buffy coat hydroxymethylation signature, i.e., whole buffy coat gDNA 5hmC signature, is correlated with the presence of cancer in a patient. The buffy coat gDNA 5hmC signature may be combined in an ensemble-type analysis, e.g., a stacked ensemble analysis, with one or more feature types, including cfDNA 5hmC signature, DNA fragment size information, such as fragment size distribution, copy number variation, and the like.

In addition to plasma, the buffy coat fraction of peripheral blood contains intact cells, such as circulating tumor cells that are widely investigated for not only cancer detection but also cancer prognosis. Yet, immune cells which make up the bulk majority of buffy coat cells have been only minimally explored for their potential utility in cancer detection. The literature over the last decade has shown that signals secreted by different types of solid tumors are sensed by the bone marrow, skewing the hematopoiesis to a myeloid bias, releasing to the periphery a heterogeneous population of immature cells collectively called MDSCs (myeloid-derived suppressor cells) which are a mix of monocytes, myeloid precursors, and neutrophils. See, e.g., Wu et al. (2014) Proc. Natl. Acad. Sci. 111:4221-26; and Casbon et al. (2015) Proc. Natl. Acad. Sci. 112:E566-575. MDSCs impair immune responses, induce angiogenesis, and promote epithelial-to-mesenchymal transition (EMT), supporting tumor growth, as explained by Marvel et al. (2015) J. Clin. Invest. 125: 3356-64. We hypothesized that the tumor-driven skew in hematopoiesis could be detected by sequencing the hydroxymethylome of circulating peripheral immune cells from the whole buffy coat, to be used as a diagnostic strategy in combination with the 5hmC profiles from plasma cfDNA and additional features, if warranted.

Epigenetic changes such as DNA methylation of CpG sites play a critical role in regulating gene expression and are globally downregulated in cancer; see Huang et al. (2014) Trends Genet. 30: 464-74. Unlike 5mC, which primarily serves as a repressive mark in the human genome, its oxidated form, 5hmC, is generally recognized as a marker for gene expression, being associated with transcriptional activation. 5hmC modifications in cfDNA have been extensively used as diagnostic biomarkers in cancer, with the potential to concomitantly identify multiple tumors, by providing information of the tissue of origin, given that specific cell types have their unique 5hmC landscape. See, e.g., Song et al. (2017) Cell Res. 27: 1231-42; Guler et al. (2020) Nat. Commun. 11: 5270; Li et al. (2017) Cell Res. 27: 1243-57; and Barefoot et al. (2021) Frontiers Genetics 12: 671057. In immune cells, as reported for tissues outside the immune system, 5hmC is preferentially enriched on tissue-specific genes and enhancers and its levels change dynamically during cell development, differentiation, and control of hematopoiesis (Nakauchi et al. (2022) Blood Cancer Discov. 3: 346-7); Tsagaratou et al. (2014) Proc. Natl. Acad. Sci. 111:E3306-E3315). Therefore, 5hmC serves as an important epigenetic mark from which cell type and disease status can be inferred.

### Example

This example is directed to the question of whether 5hmC profiles of buffy coat gDNA is altered in cancer, particularly in breast, colorectal and lung cancer. We assessed the cancer classification potential of buffy coat 5hmC features alone and in combination with cfDNA-derived features. We sequenced the genomic DNA of buffy coat and the plasma cfDNA from the same individuals in order to build predictive models that yielded cancer classification.

### A. Study Design and Methods:

### (i) Clinical Cohorts and Study Design:

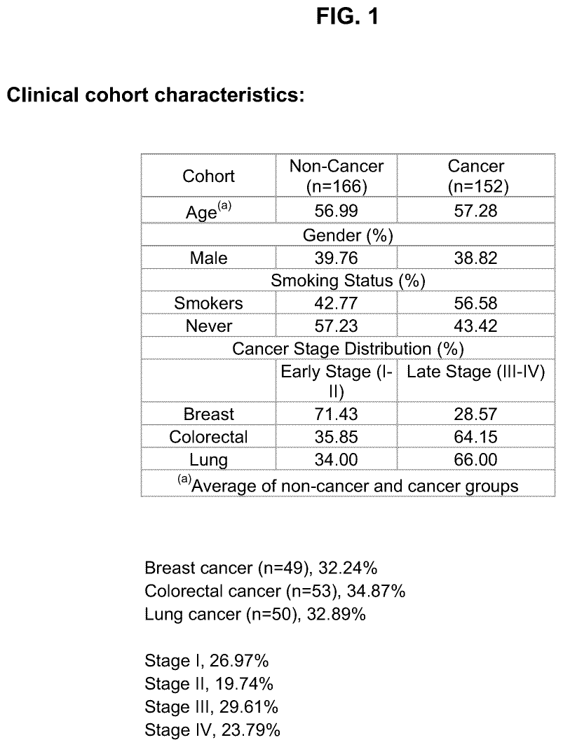

To assess the potential impact of cancer on buffy coat 5hmC profiles, peripheral blood samples were collected from 318 male and female subjects, minimum age 45 years old, with 152 cancer samples and 166 non-cancer controls. As indicated in FIG. 1, all patients in the cancer cohort had a confirmed pathologic diagnosis of breast (n=49), colorectal (n=53), or lung (n=50) cancer of any subtype at the time of biopsy or surgical resection. 46.7% of the cancer cohort had early-stage (stages I and II) disease. The percentage of early-stage cancer was 71.4%, 35.8% and 34% for breast, colorectal and lung, respectively. The cancer cohort was cancer-treatment naïve. The non-cancer cohort was negative for any form of cancer. Neither cohort was being treated with immunomodulators at the time of blood collection or at any time during the prior 12 months prior. Blood samples from cancer subjects were collected prior to biopsy or surgical resection.

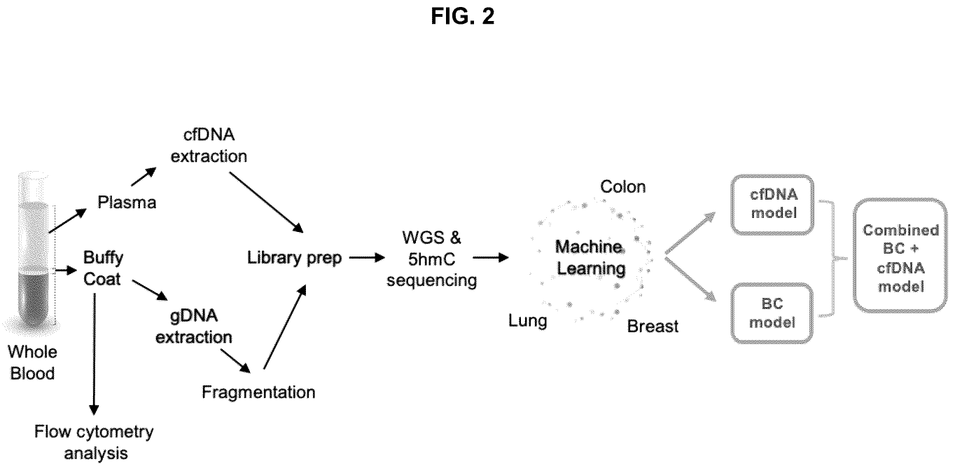

A single blood draw from each individual was processed to isolate BC gDNA and plasma-derived cfDNA, which were then used as input material for WGS and 5hmC sequencing and subsequent analysis to compare and classify cancer and non-cancer samples. The process is schematically illustrated in FIG. 2.

### (ii) Blood Collection and Processing:

A single blood draw from each individual was processed to isolate buffy coat gDNA and plasma-derived cfDNA, which were ultimately used as input material for WGS and 5hmC sequencing and subsequent analysis to compare and classify cancer and non-cancer samples, as will be explained infra. Two Cell-Free DNA BCT® Streck tubes containing 10 ml of whole peripheral blood each were obtained per individual, by routine venous phlebotomy, according to the manufacturer's instructions. Streck tubes were kept at 15-25° C. and processed within 96 hours of phlebotomy by centrifugation at 1,500 rcf for 10 minutes with the brake off at room temperature. The top layer containing plasma was collected and transferred to a new tube, and the layer of buffy coat was then carefully transferred to a 50 ml conical tube.

(iii) Plasma Isolation and cfDNA Extraction:

Plasma collected as described above was spun at 3,000 rcf for 10 min with the brake on at room temperature. The supernatant was transferred to two 5 ml conical tubes and stored at −80° C. cfDNA was extracted from 4 mL of plasma using MyOne® (ferrimagnetic) Silane Beads cfDNA isolation kit (Thermo Fisher) following manufacturer's instructions, in a HAMILTON STAR automated liquid handler (HAMILTON Co., Reno NV). During this procedure, plasma was incubated with Proteinase K and 20% SDS at 60° C. for 20 minutes followed by cooling. Next, the cfDNA was bound to the magnetic beads and washed with a Thermo Fisher Scientific proprietary wash buffer and with 80% ethanol. Finally, cfDNA was eluted with elution buffer, quantitated using Molecular Devices' Spectramax® Plate Readers and the Quant-iT™ PicoGreen® dsDNA quantitation assay (Thermo Fisher), and stored at −20° C. TapeStation® 4200 capillary electrophoresis (Agilent Technologies, Santa Clara, CA) was employed to ensure the absence of contaminating high molecular weight DNA emanating from white blood cell lysis.

### (iv) Buffy Coat Isolation and Genomic DNA Extraction:

3 volumes of RBC Lysis Solution (QIAGEN, red blood cell lysis buffer) were added to the pooled buffy coat samples, followed by two rounds of vortexing and incubation on ice for 10 minutes. Samples were spun at 400 rcf for 10 minutes at 4° C., the supernatant was removed, and the cell pellet was washed twice with 1× PBS with 2% FBS. The cell pellet was then resuspended in 1 mL 100% FBS. 25 μL of this cell suspension was used for FACS staining, and 50 μL was transferred to a 1.5 mL Eppendorf tube and spun at 400 rcf for 5 minutes at room temperature, the supernatant was removed, and the cell pellet was stored at −80° C. Genomic DNA was extracted from cell pellets stored at −80° C. using the DNeasy® Blood & Tissue Kit (QIAGEN), following the manufacturer's instructions. gDNA eluates were quantified using SpectraMax® iD3 (Molecular Devices). 100 ng of gDNA were sonicated to a modal 150 bp size using an ME220 focused ultrasonicator (Covaris). The sonicated DNA fragments were verified by TapeStation® 2200 dsDNA high sensitivity assay (Agilent).

### (v) Granulocyte and Monocyte Enrichment:

Nine tenths of the isolated buffy coat was used to enrich granulocytes and monocytes using immunomagnetic cell isolation kits, following the manufacturer's instructions. Granulocytes were enriched using the EasySep® HLA Chimerism Whole Blood CD15 Positive Selection Kit (cat: 17881, StemCell), followed by monocytes isolation using the EasySep Human CD14 Positive Selection Kit II (cat: 17858, StemCell). The isolated cells were spun at 400 rcf for 5 minutes at room temperature and resuspended in 500 μL of 1× PBS with 2% FBS and 1 mM EDTA. 25 μL of this cell suspension was used for FACS staining to confirm at least 85% of specific cell enrichment. The remaining cell suspension was spun at 400 rcf for 5 minutes at room temperature, the supernatant was removed, and the cell pellet was stored at −80° C. until gDNA processing.

(vi) Flow cytometry:

Cells were incubated with 2.5 μL Fc block (BioLegend) for 5 minutes in a 96-well plate and then stained with fluorescent antibodies at room temperature in the dark, 50 μL total volume. The antibodies were anti-CD45-PECy5 clone HI30, anti-CD14-AF700 clone 63D3, anti-CD15-FITC or anti-CD15-PE clone H198 and anti-CD3-APC-Cy7 clone OKT3 (all obtained from BioLegend). After 15 minutes, the cells were washed twice with FACS buffer (PBS, 2% FBS, 1 mM EDTA) and spun at 1500 rpm for 5 minutes at room temperature. The cell pellet was resuspended in FACS buffer and cell analysis was performed on a NovoCyte® Advanteon® Flow Cytometer (Agilent). Data points were analyzed using the NovoExpress® software (Agilent).

(vii) 5hmC Enrichment Assay:

5hmC enrichment and subsequent sequencing libraries were prepared as described previously (Guler et al. (2020), supra) using the “5hmC-Seal” method of International Patent Publication WO 2017/176630 to Quake et al., Song et al. (2011) 29: 68-72, and Han et al. (2016) Mol. Cell 63:711-19, the disclosures of which are incorporated by reference herein. Briefly, hMe-Seal is a low-input, whole-genome 5hmC sequencing and enrichment method based on selective chemical labeling, in which β-glucosyltransferase (B-GT) is used to selectively label 5hmC with a biotin moiety via an azide-modified glucose for pull-down of 5hmC-containing DNA fragments for sequencing. In implementing hMe-Seal in the present case, the normalized buffy coat gDNA and the normalized cfDNA were ligated to sequencing adapters, followed by selective labeling of 5hmC with β-GT, and affinity enrichment via selective pull-down of DNA fragments containing biotin-labeled 5hmC by binding to Dynabeads® M270 streptavidin (Thermo Fisher). PCR was then carried out directly on the beads to minimize sample loss during purification.

(viii) Library Preparation and Sequencing:

Adapter-ligated DNA fragments were prepared for library construction using the KAPA Hyperprep® kit (Roche) according to the manufacturer's instructions. All libraries were quantitated using the Qubit® dsDNA High Sensitivity Assay (Thermo Fisher Scientific) and normalized in preparation for sequencing. 75 base-pair paired-end sequencing was performed on a NovaSeq6000 instrument (Illumina). Sequencing data were collected with NovaSeq Control Software v1.7.0 (Illumina), as explained in part (v) of the next section.

### B. Bioinformatic Analysis:

### (i) Raw Data Processing and Alignment (Hg38):

Raw data processing and demultiplexing were performed using bcl2fastq Conversion Software (Illumina, Inc.) to generate FASTQ output for each sample. Sequencing reads were aligned to the human genome build 38 reference genome using the BWA-MEM2 algorithm (Li et al. (2013) Arxiv doi:10.48550/arxiv.1303.3997). Sequencing metrics were computed with Picard (http://broadinstitute.github.io/picard/) to assess the quality of the sequencing data.

### (ii) Differential Analysis:

To identify genes with differential 5-hydroxymethylation between the cancer and non-cancer cohorts, we first removed genes that mapped to non-autosomes along with genes with CPM (counts per million)>3 in fewer than 10 samples. Following TMM (trimmed mean of M values; see Robinson et al. (2010) Genome Biol 11: R25 normalization of gene representation distributions, differential analysis was performed using the software package edgeR (empirical analysis of digital gene expression data in R). p-values were adjusted for multiple comparisons using the Benjamini-Hochberg method, to decrease the overall incidence of false positives.

(iii) Gene Set Enrichment Analysis:

Gene set enrichment analysis was done using java software GSEA v3.0 (http://www.gsea-msigdb.org/gsea/index.jsp). Log 2 fold change of CPM (counts per million) between cancer and non-cancer was used as input for the pre-rank gene list tool GSEAPreranked, with default setting except following: “Enrichment statistics”=classic, “Normalization mode”=NONE. Molecular Signatures Database (MSigDB) gene sets C7 (immunologic signature) and C8 (cell type signature) gene sets were searched.

### (iv) Statistical Analysis:

All statistical analyses were done using R (https://www.r-project.org/) unless otherwise stated. For sensitivity comparison between two models, McNemar's chi-squared test for count data was applied (see Alan Agresti (1990), “Categorical Data Analysis (New York: Wiley, 1990, pages 350-354; and Nilima et al. (Mar. 2019) J. Clin. Diagnost. Res. 13(3): YG01-YG04). Overlap between two sets of gene lists was assessed using a hypergeometric test, and the two-sample t-test was used to compare relative induction percentage of blood subpopulations.

### (v) Algorithm Training:

Sequencing data from 5hmC and WGS was produced using NovaSeq® Control Software v1.7.0 (Illumina, Inc.). Raw data processing and demultiplexing were performed using bcl2fastq Conversion Software (Illumina, Inc.) to generate sample-specific FASTQ output. Sequencing reads were analyzed by a computational pipeline implemented as a Nextflow® script, which aligns the reads to the human genome reference build 38 (GRCh38 or Hg38) using the BWA-MEM2 algorithm (Anaconda.org, version 2.2.1). Metrics were computed by the pipeline via Picard to assess the quality of the sequencing data. Samples passing quality control metrics were placed into the two datasets to be used for training and validation. The quality control failure rate for the set of validation samples was 1.94%. Noncancer samples in the training data were matched to various clinical features such as age, sex, body mass index, and smoking status. The machine-learning classification algorithm was trained as follows: each sample included in the training dataset was analyzed with the bioinformatics pipeline as already described. The pipeline divided the genome into functional regions pertaining to annotated gene bodies, enhancers, CpG islands, CCCTC-binding factor sites, promoters, and 3-prime untranslated regions from Gencode human annotation version 31 (GRCh38.p12), and then counted, with the number of 5hmC library read pairs mapped to each region, correcting for differences in coverage using counts per million mapped reads. In addition, feature sets incorporating copy number across 100 kb bins and fragment size variation across the genome were created using the WGS data. Elastic net logistic regression algorithms were built using the R package glmnet for each of the feature sets, with the elastic net mixing ratio α and the regularization parameter λ optimized using 10-fold cross validation. To simulate how well the algorithm would perform on new data, the algorithm was assessed using 20-fold cross validation enabling 5% of samples to be held out from training and instead used for validation. The classification probability threshold used to calculate sensitivity and specificity was determined by setting a threshold that resulted in x % specificity of the noncancers in the training data within each cross validation fold.

### C. Results:

### (i) Identification of Differentially Hydroxymethylated Genes in Buffy Coat Cancer Samples:

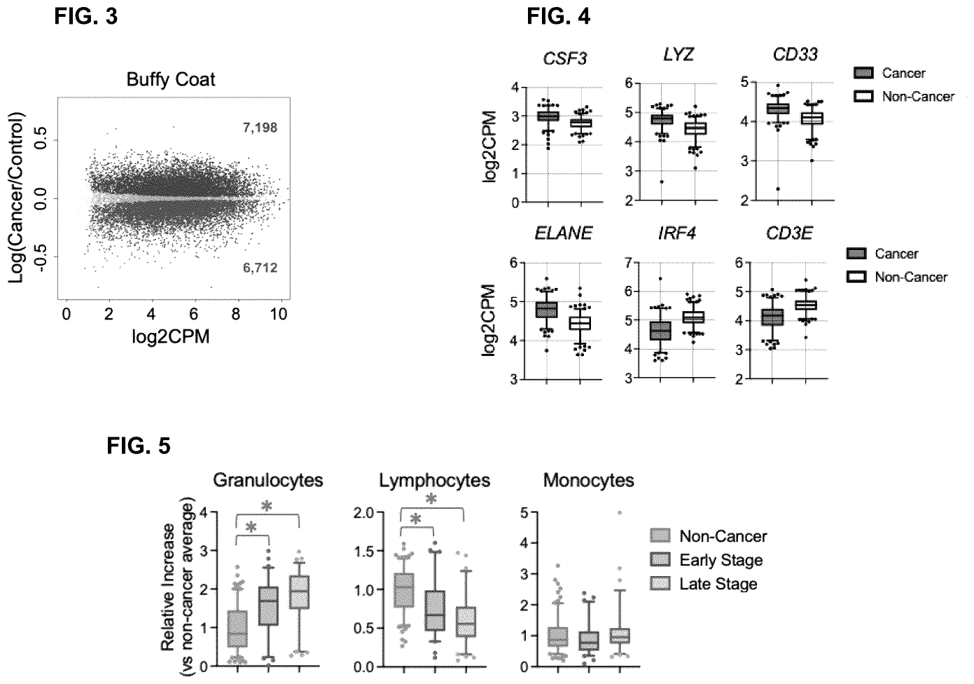

To evaluate the use of a circulating white blood cells hydroxymethylome for cancer prediction, 5hmC profiles of buffy coat-derived gDNA obtained from individuals with cancer were compared to those associated with non-cancer controls. The comparison resulted in 7,198 hyper-hydroxymethylated genes (“Hyper DhMG” in Table 1 below) and 6,712 hypo-hydroxymethylated genes (“Hypo DhMG” in Table 1 below) with FDR≤0.05; see the MA plot of FIG. 3.

TABLE 1

| Cancer Type | Hyper-DhMG | Hypo-DhMG |
|---|---|---|
| All cancers | 7198 | 6712 |
| Breast | 4192 | 3580 |
| Colon | 6470 | 6612 |
| Lung | 7125 | 6623 |

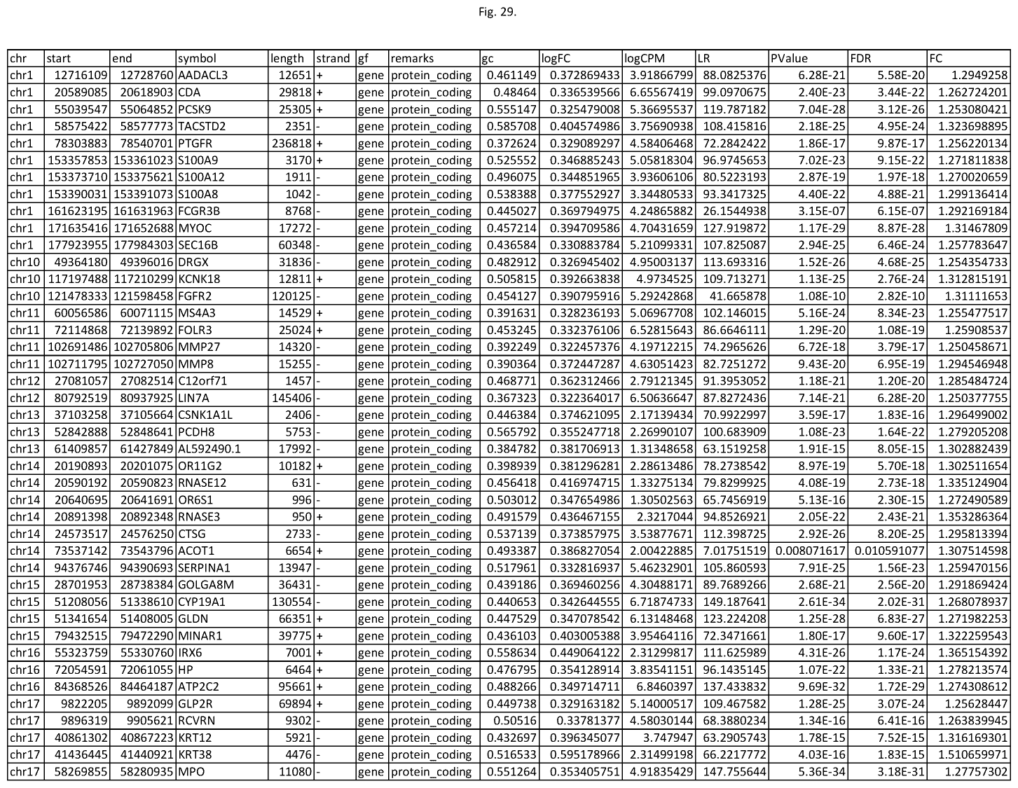

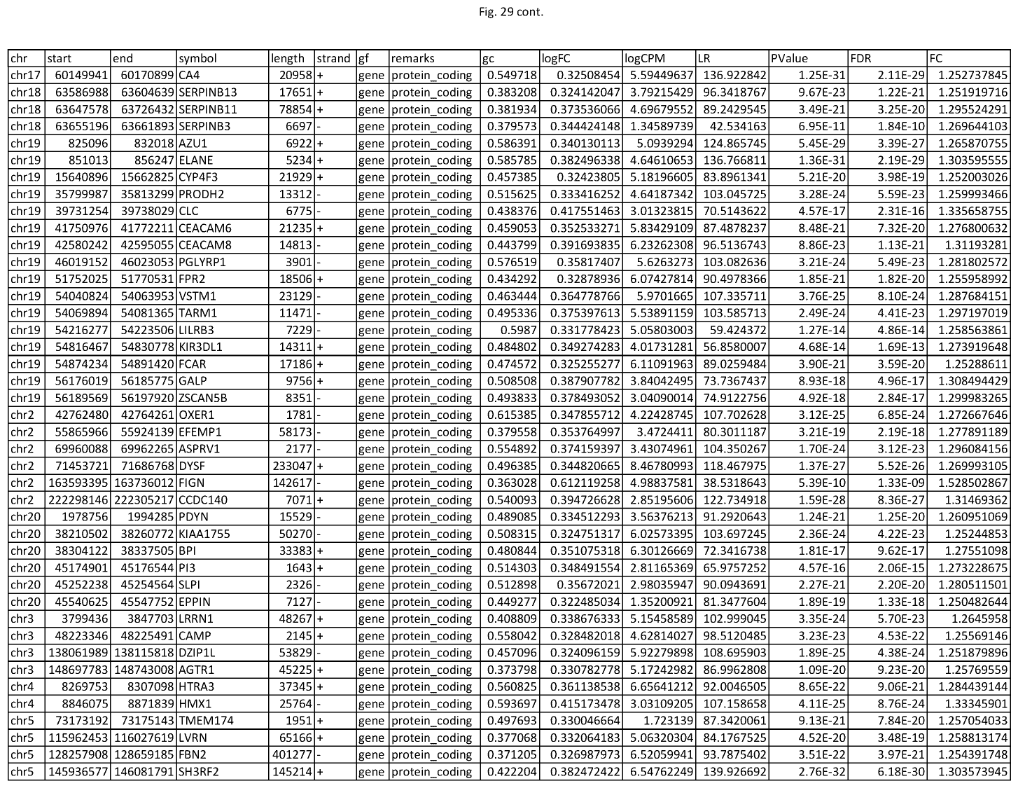

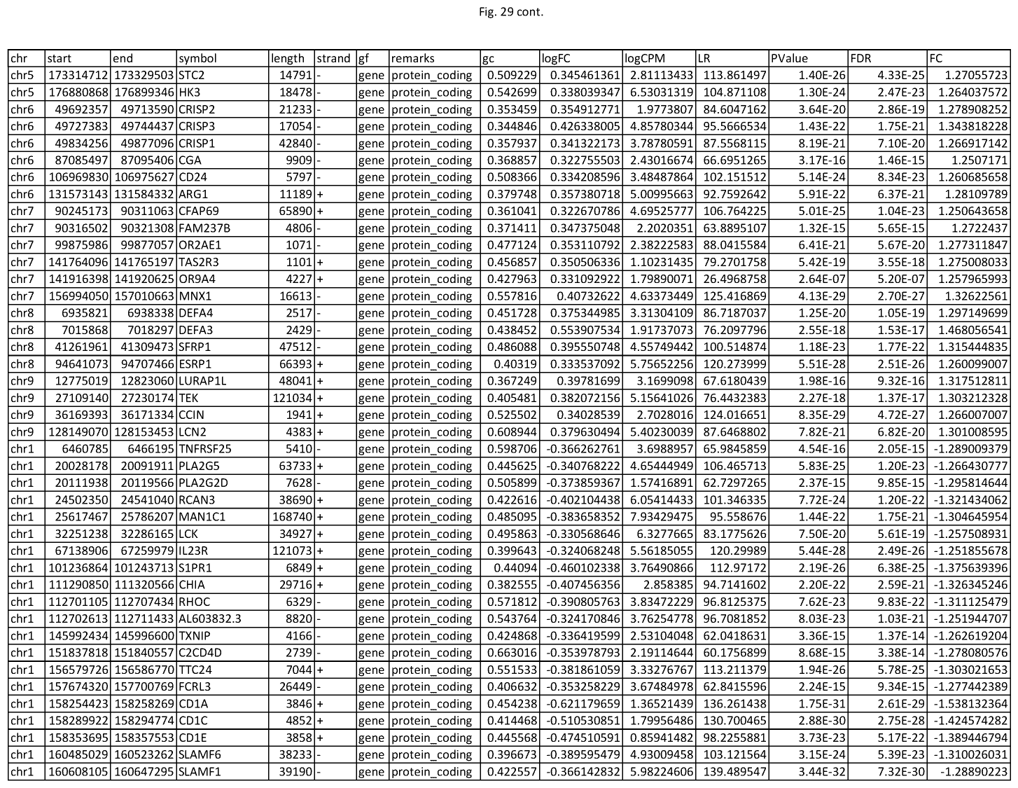

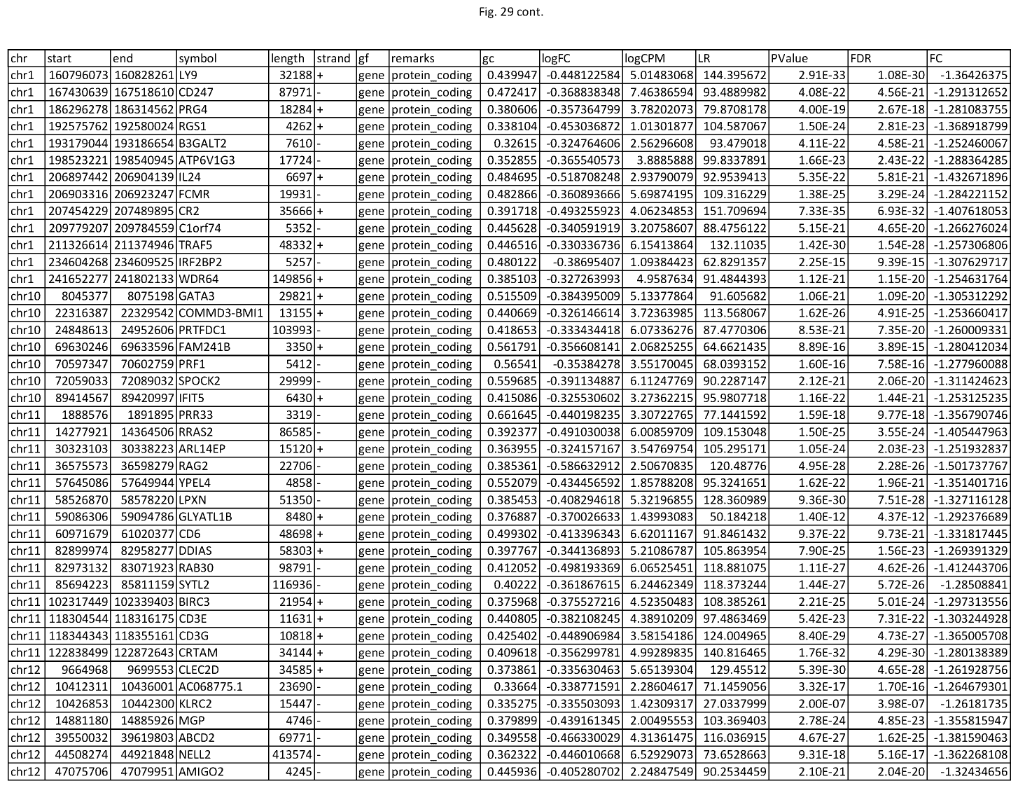

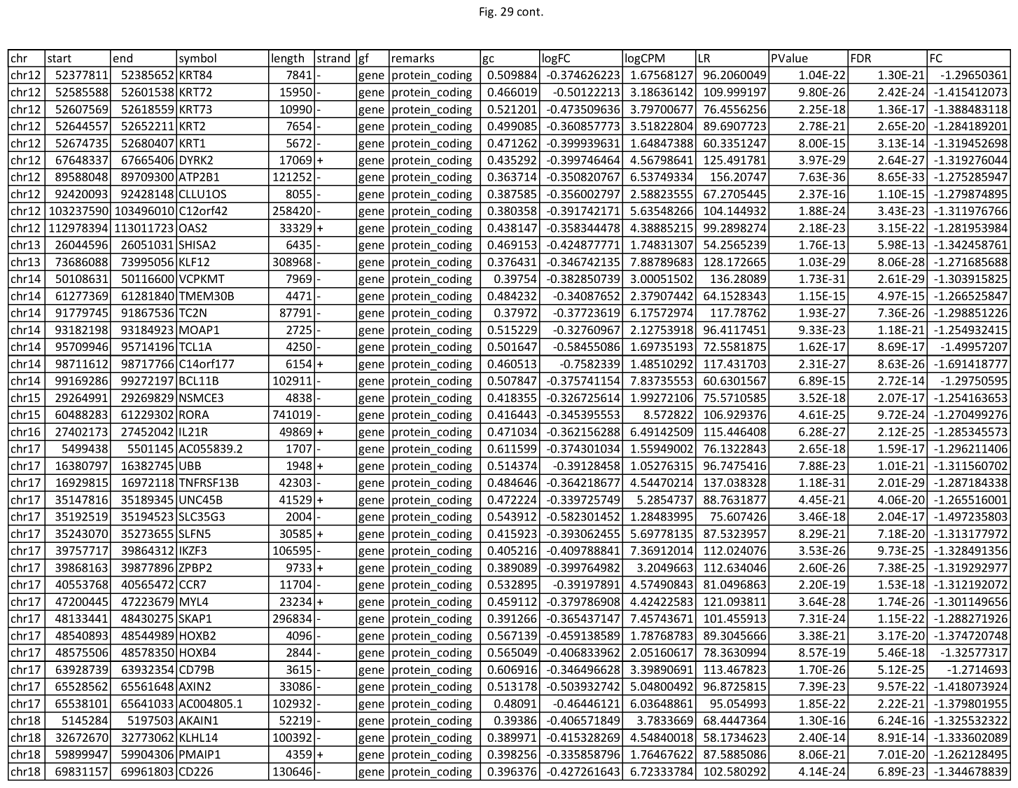

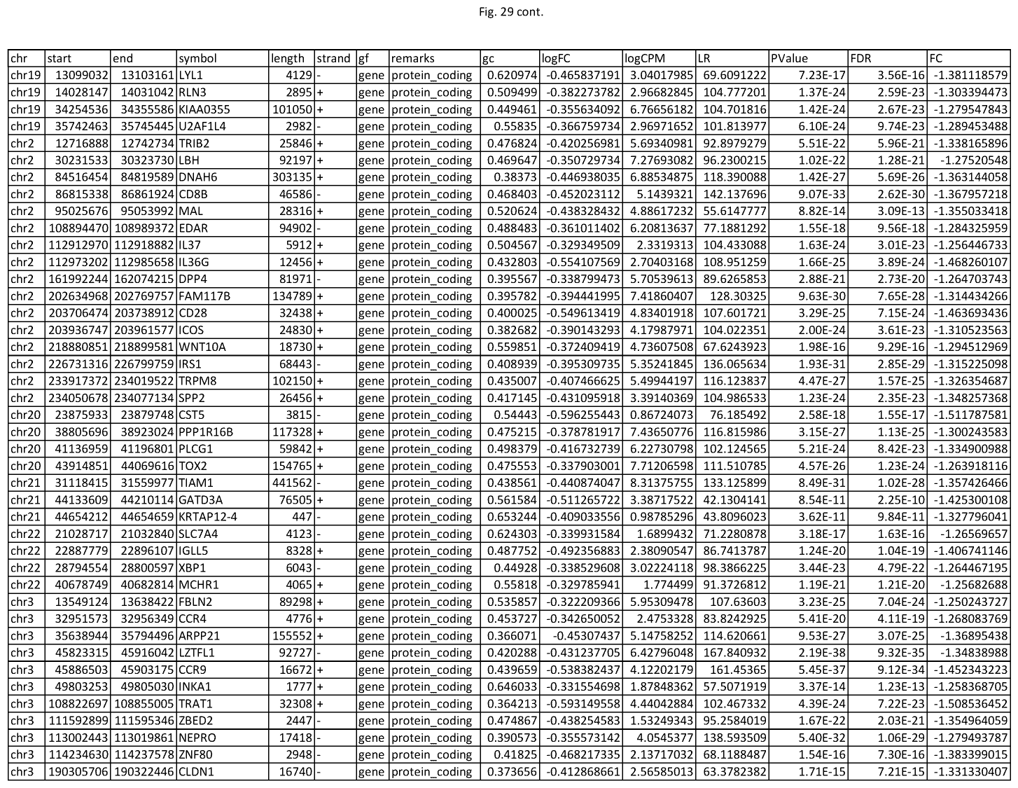

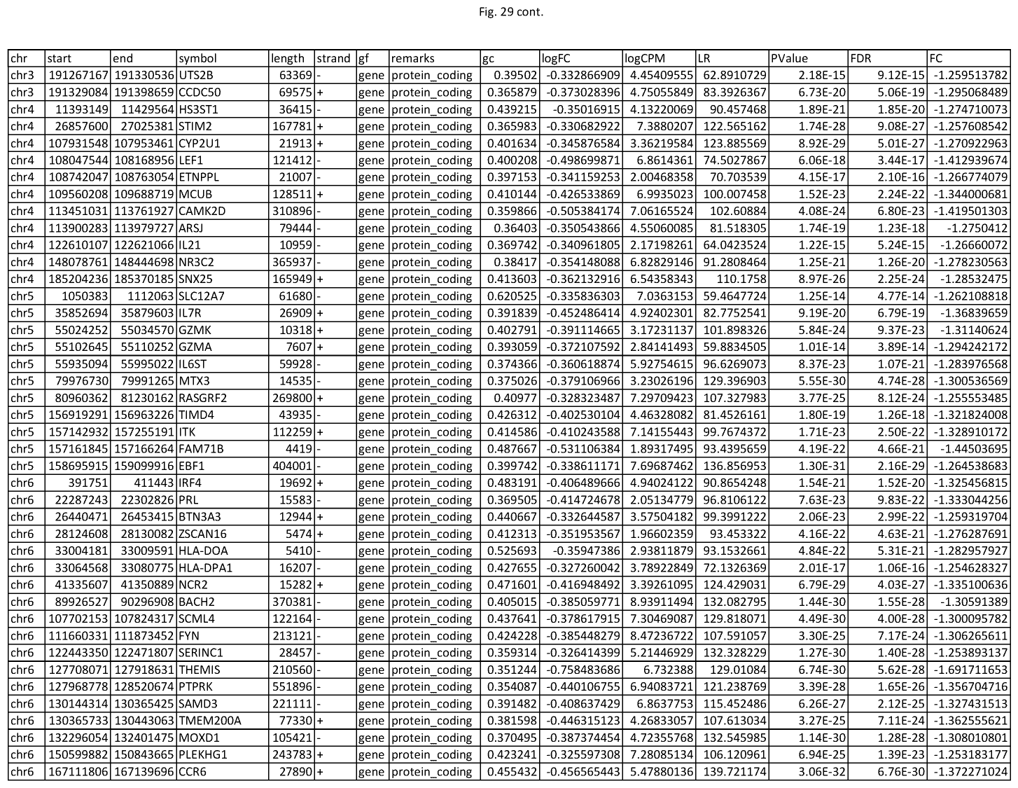

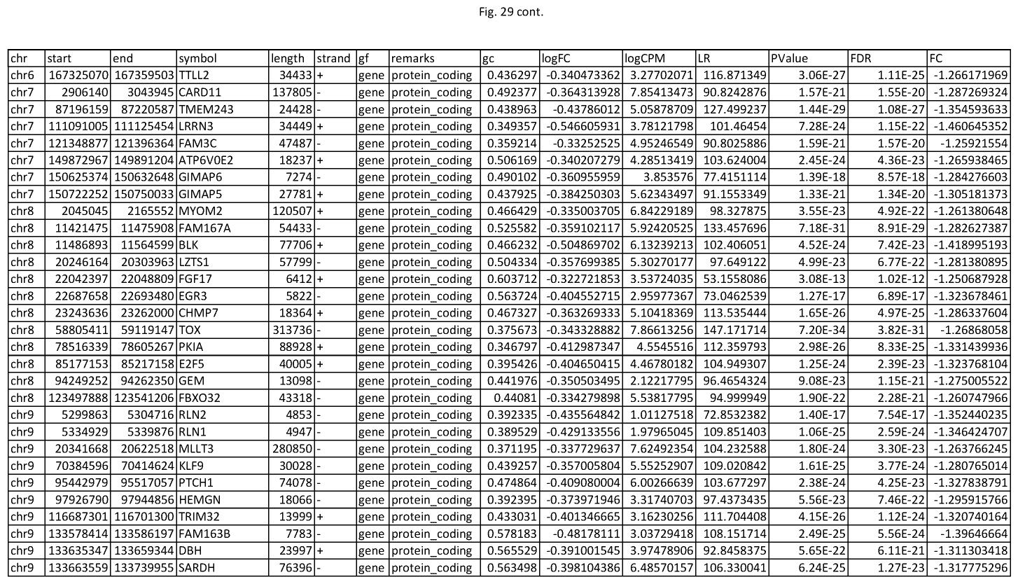

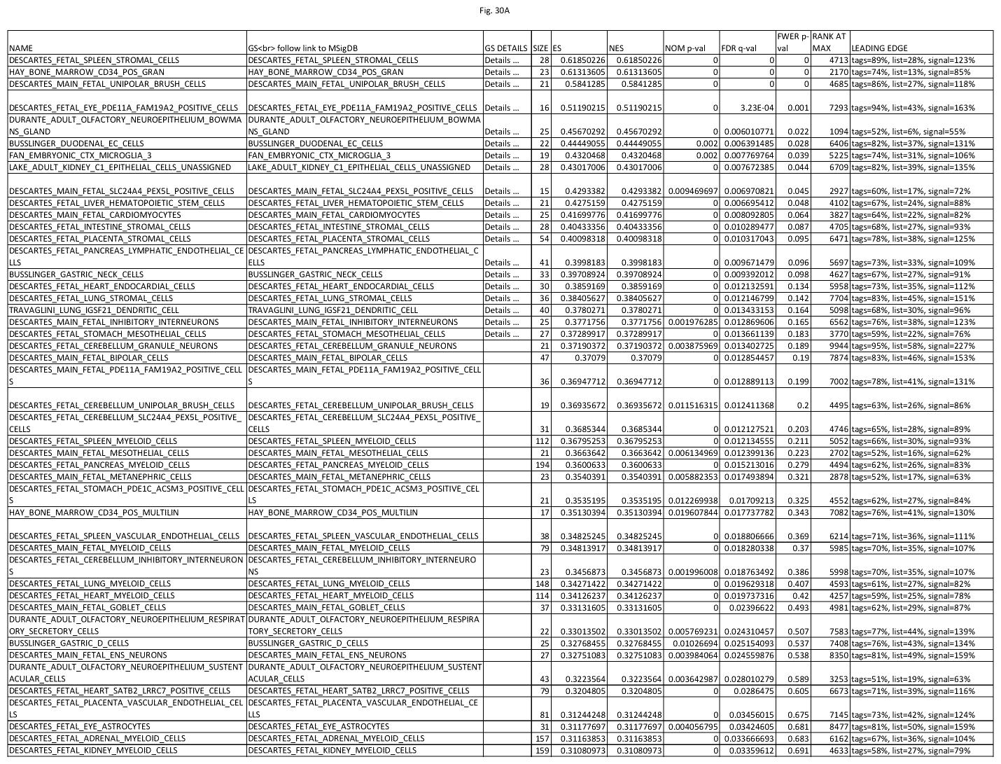

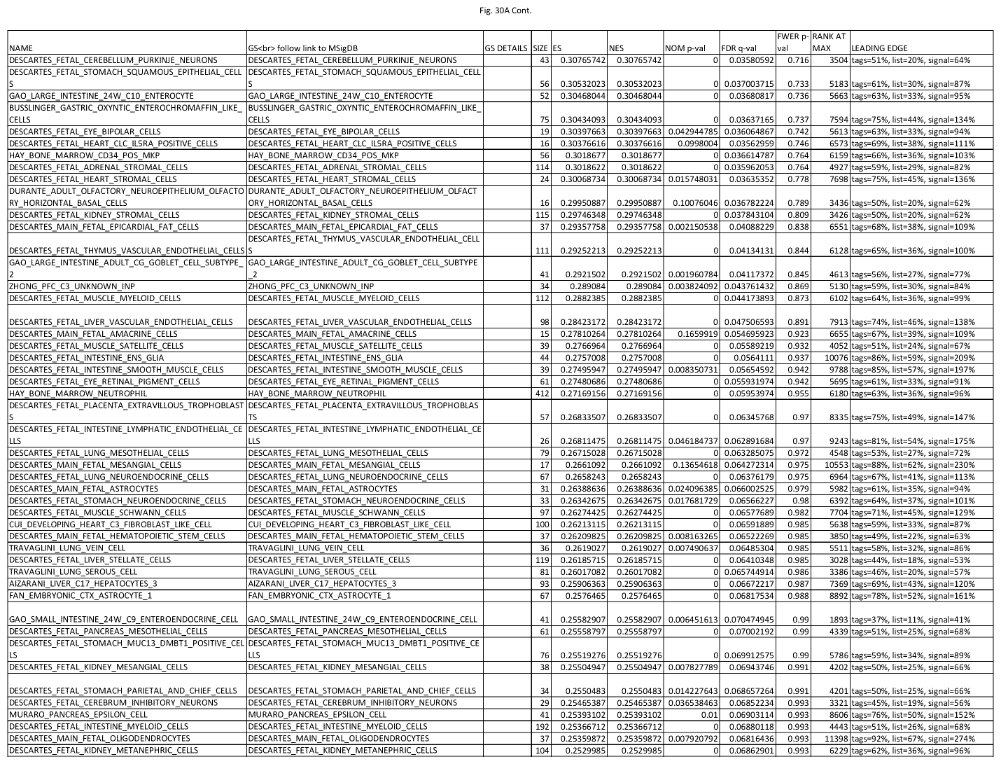

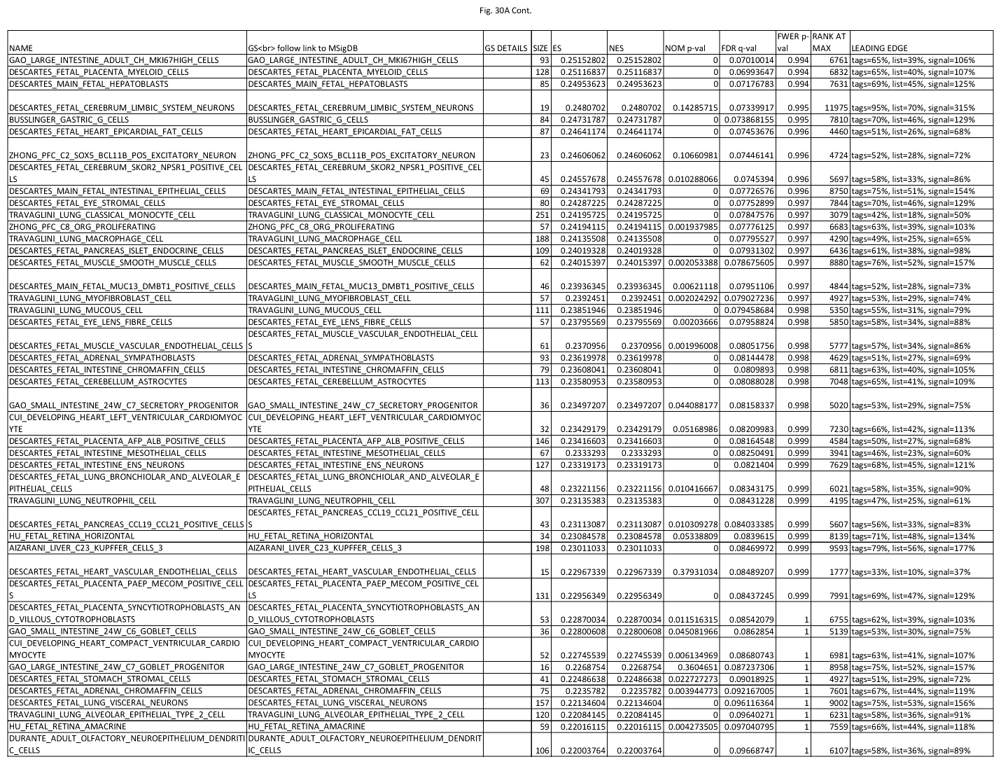

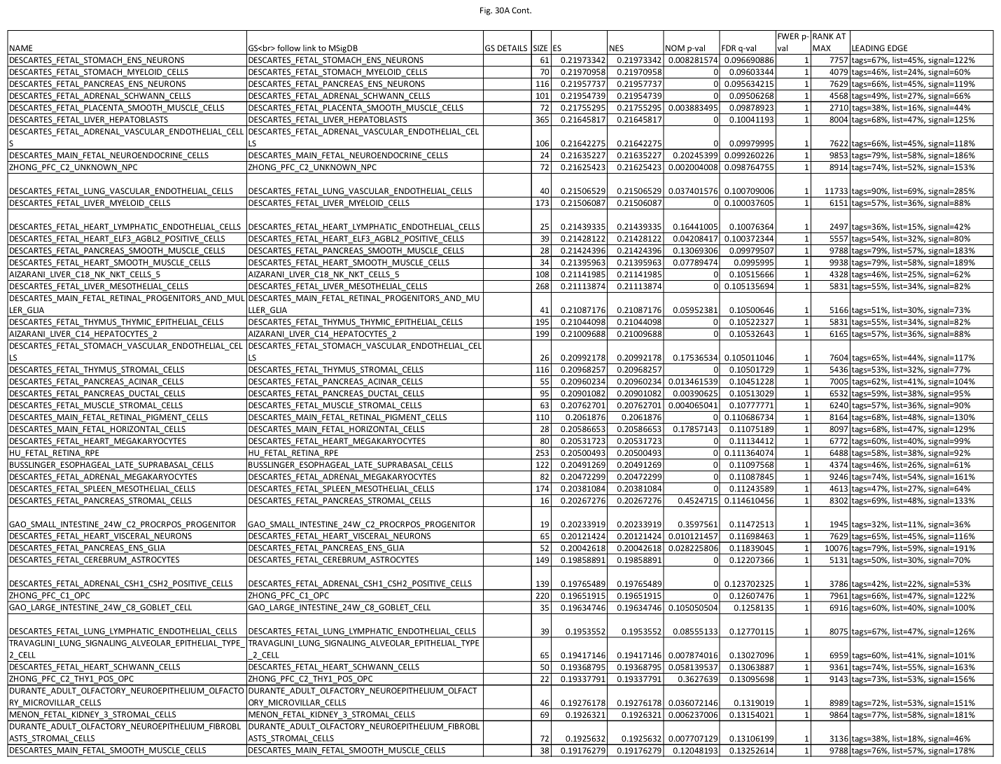

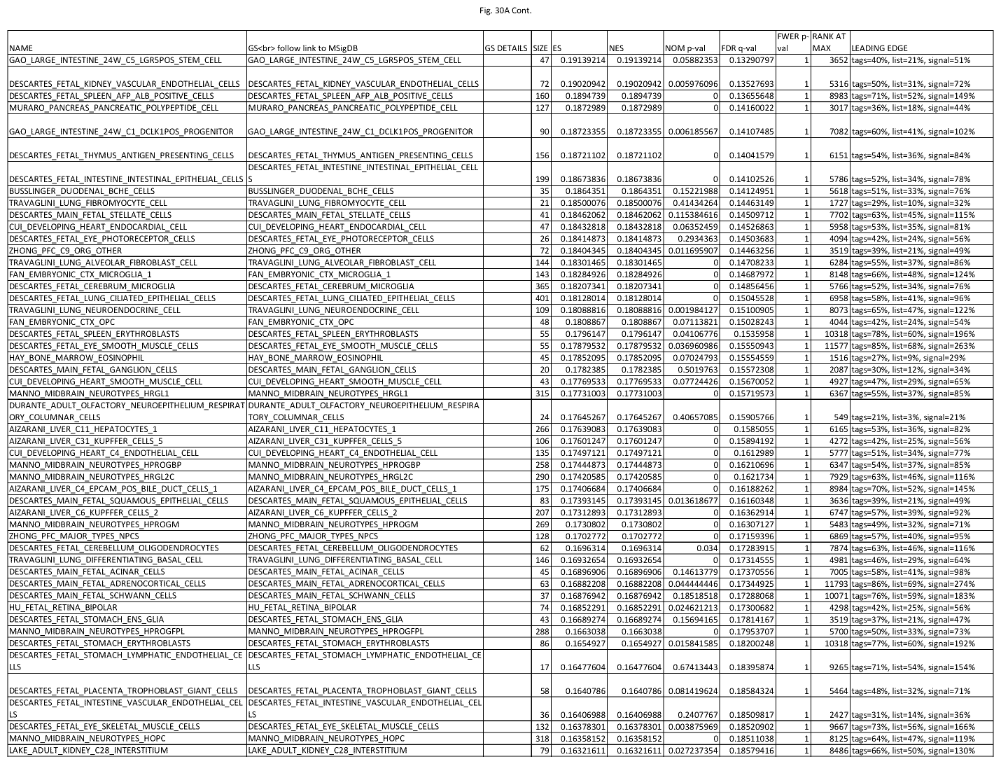

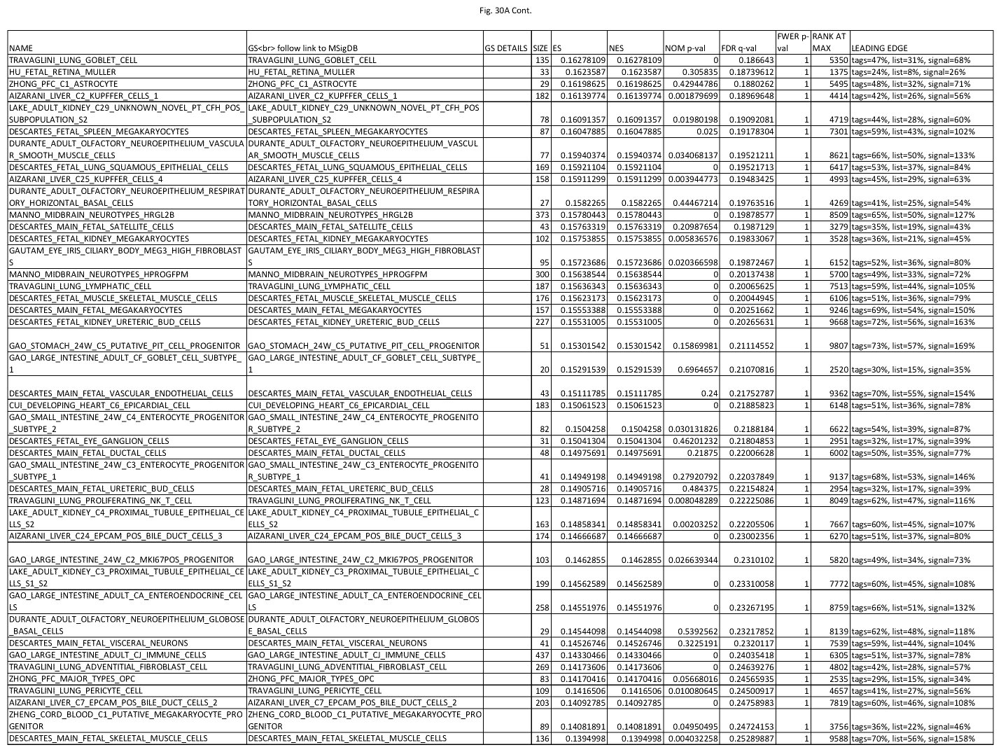

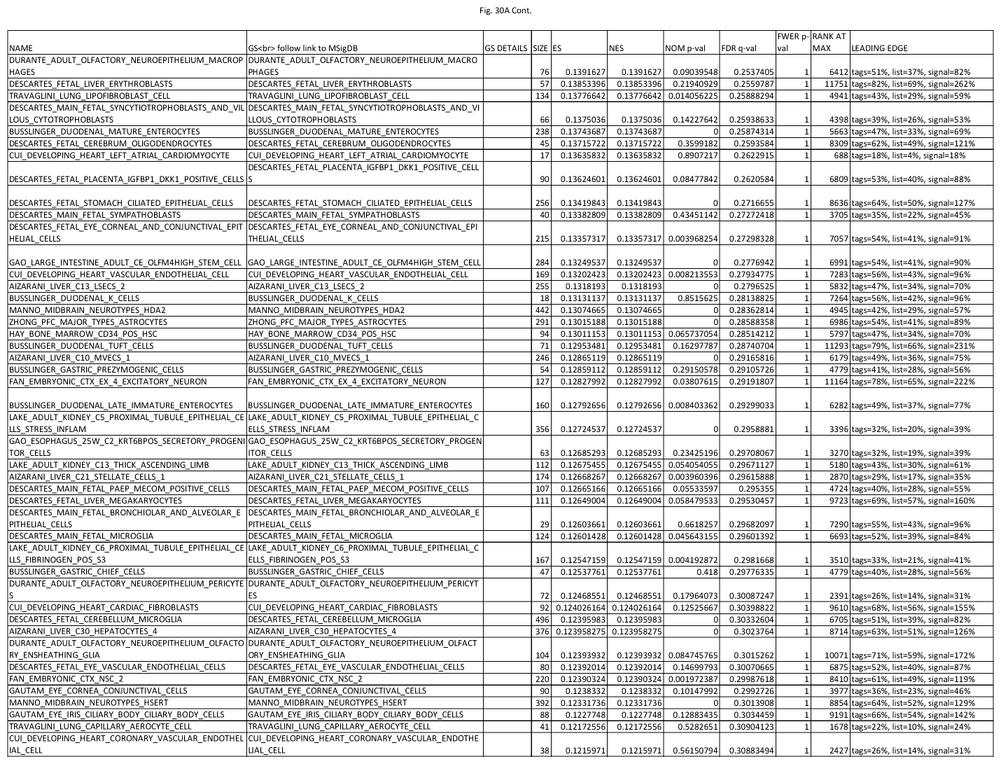

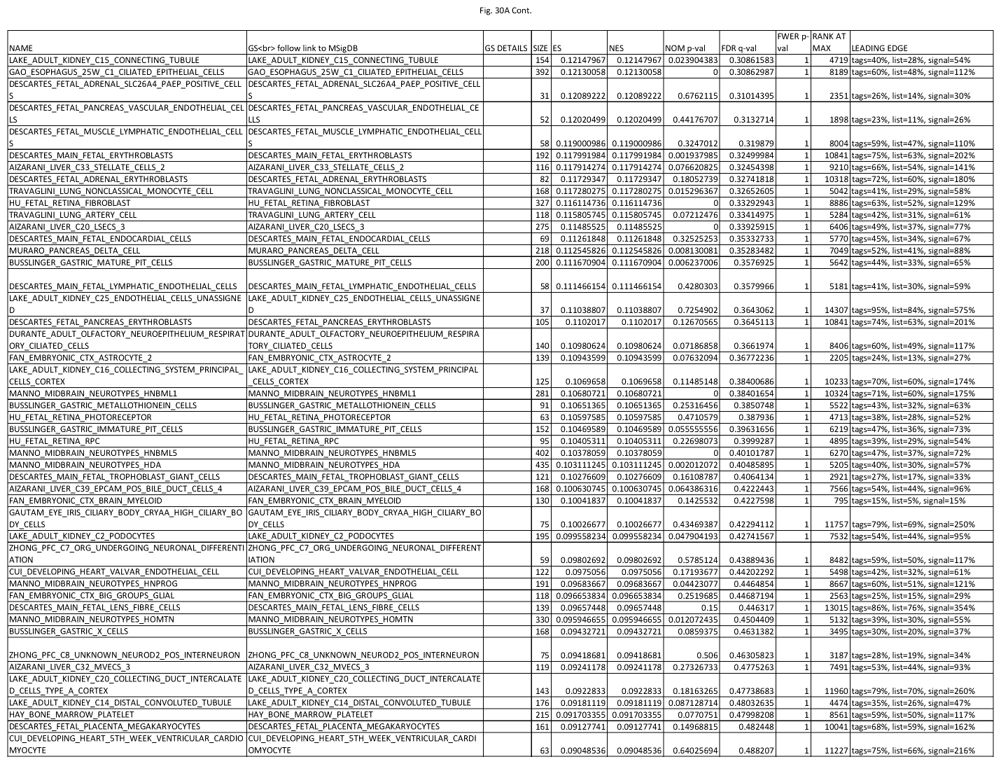

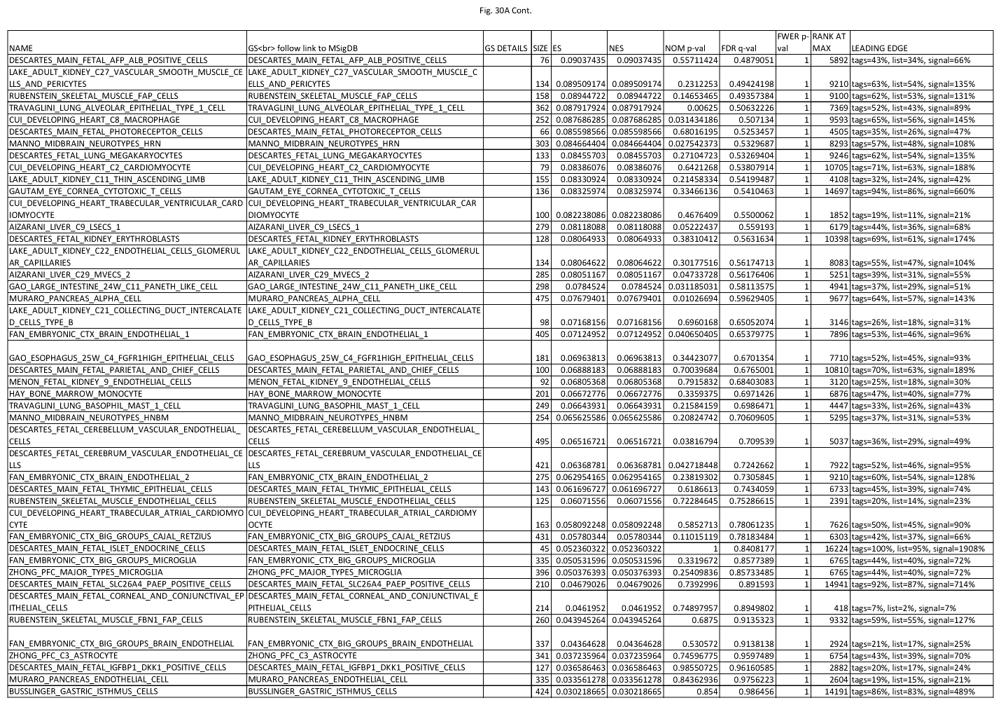

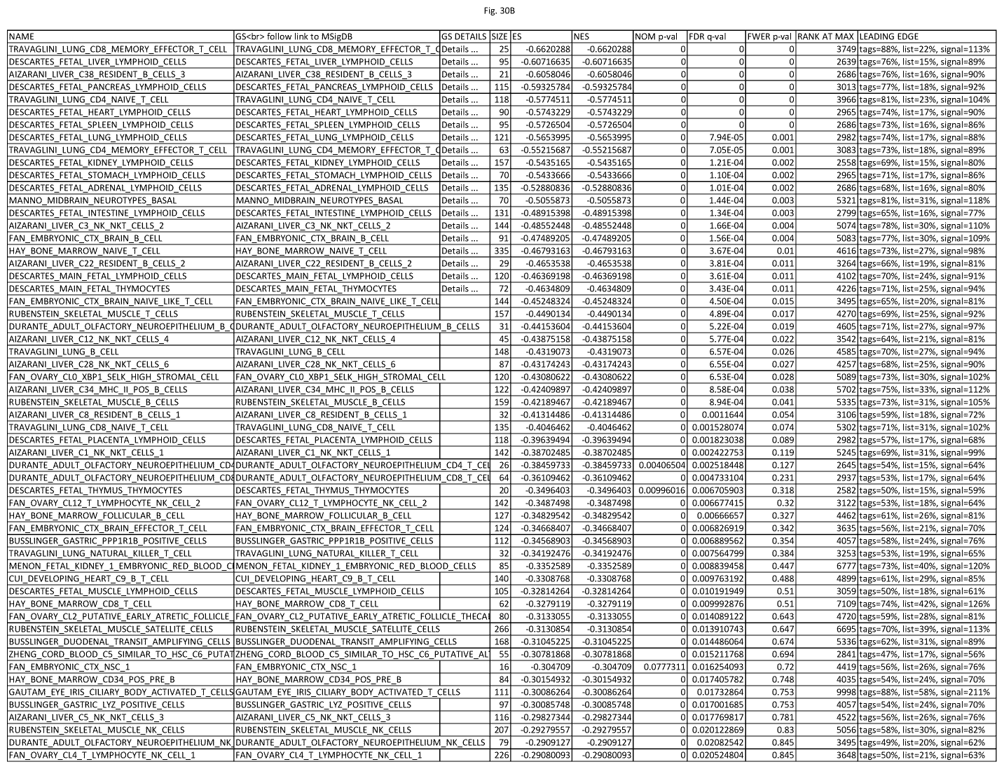

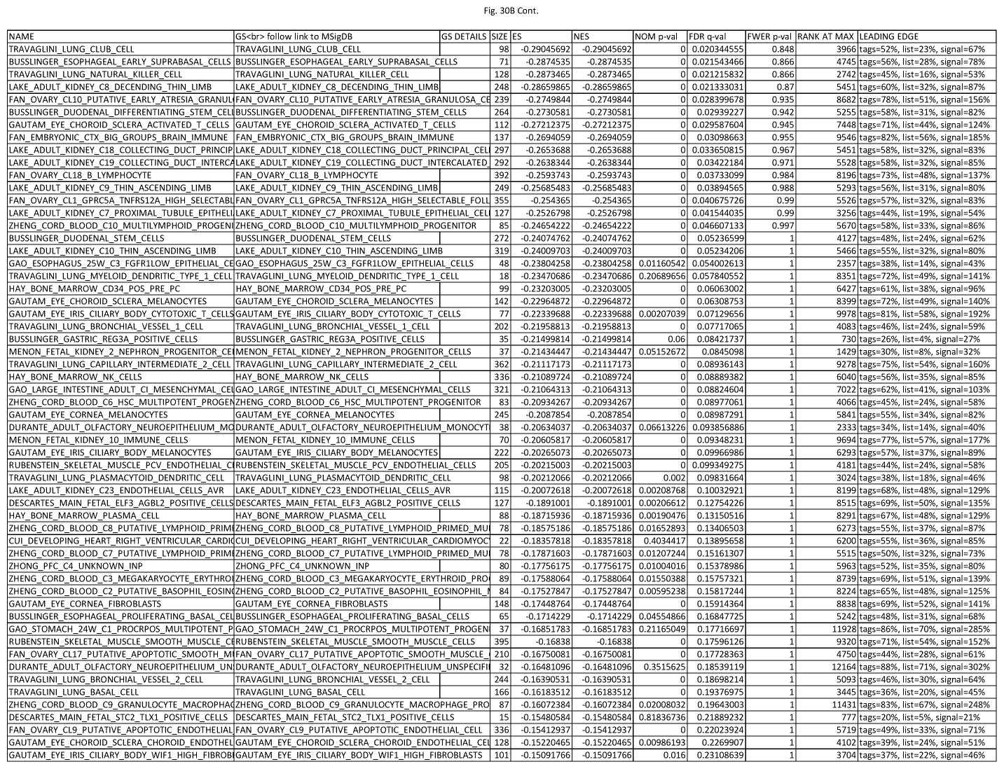

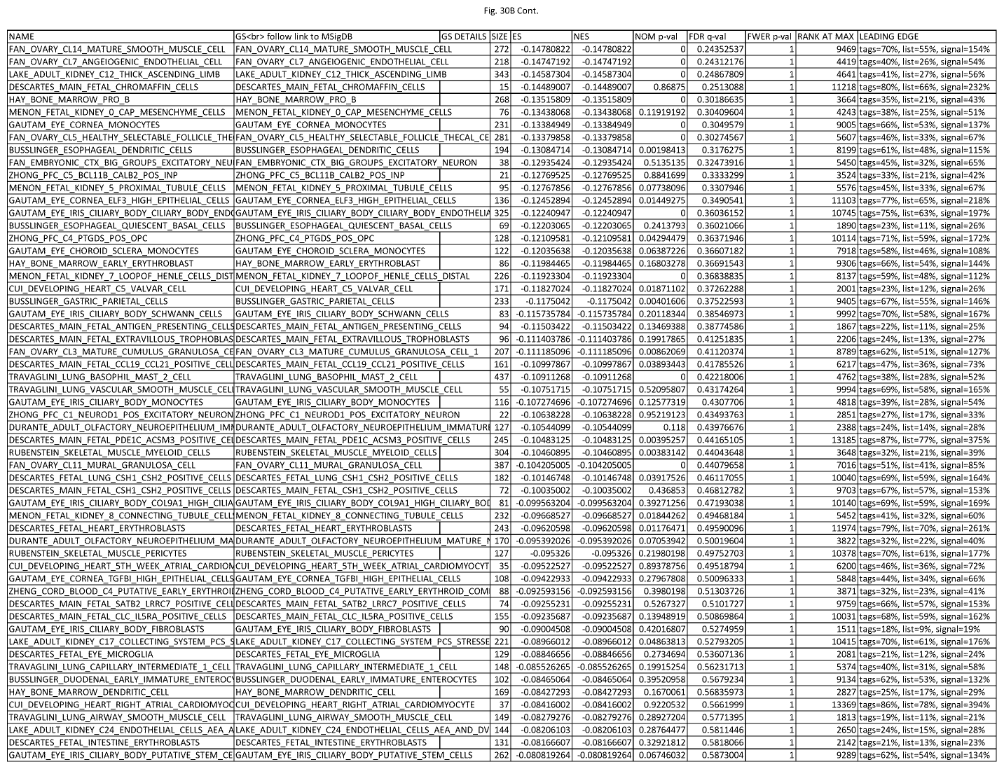

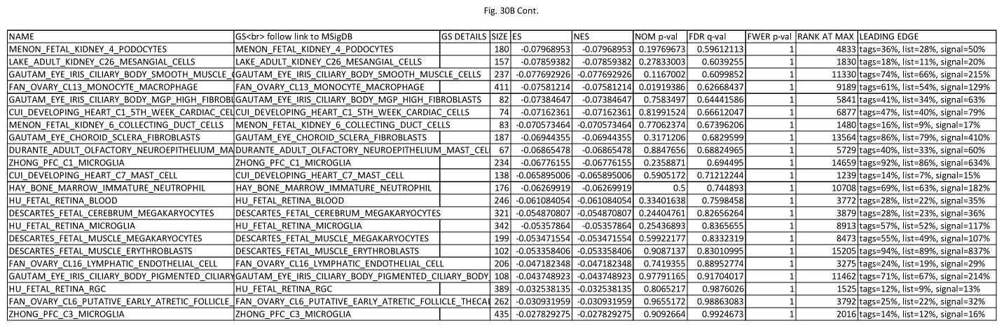

Similarly, comparison of individual cancer types separately to non-cancer controls yielded thousands of genes with differential hydroxymethylation showing that the differences observed are not driven by single cancer type but are present in three cancer types investigated. Among the genes with increased 5hmC in cancer were myeloid/neutrophil-specific genes (CAMP, CD33, ELANE, FCGR3B [encoding CD16], MNDA, SLPI), secretory vesicles/granules (ARG1, CEACAM1, CXCL1, HP, LTF, LYZ, PGLYRP1), and inflammatory genes (CSF3 [encoding G-CSF], CXCL6, FPR1, IL18, IL22, SERPINA1). The genes with decreased 5hmC included lymphocyte activation and differentiation genes such as BLK, CD3E, CCR7, CD28, FYN, GATA3, ICOS, IGLL5 and IRF4 (FIG. 4; also see the table of FIG. 29).

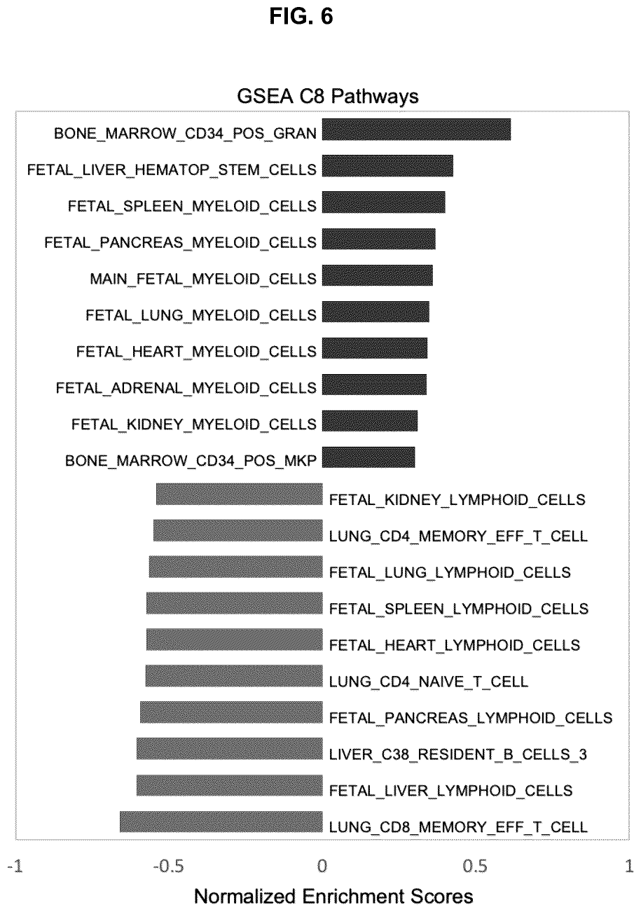

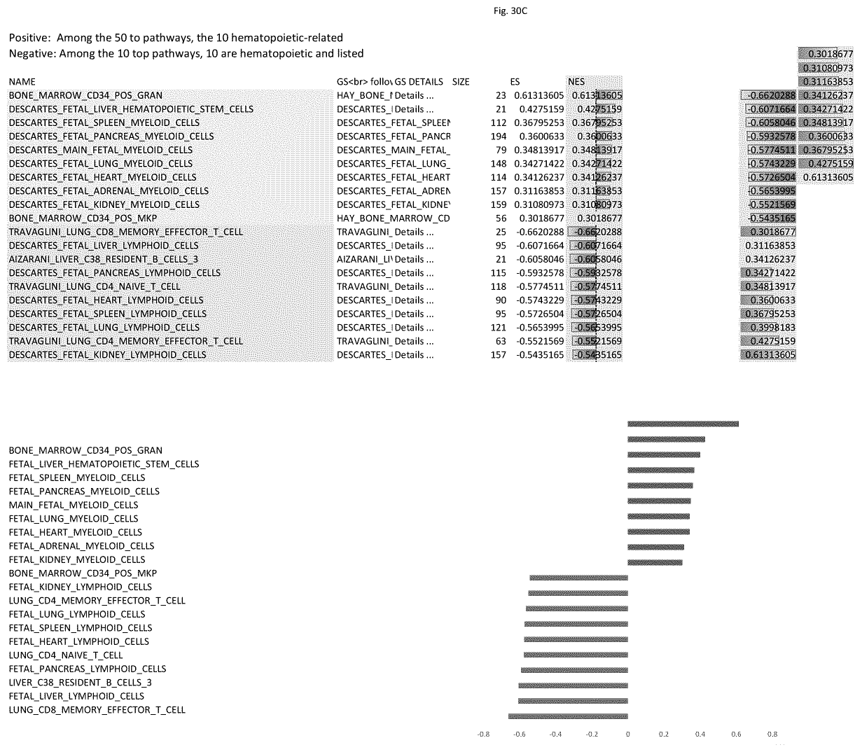

To gain insight into the biology of the differentially hydroxymethylated genes (DhMGs) identified, we performed gene set enrichment analysis (GSEA) as described in part (iii) of Section B, above. Examination of GSEA cell type signature (C8) gene sets related to hematopoiesis revealed that 8 of the top 10 pathways with increased 5hmC in cancer were myeloid pathways, while all 10 top pathways with decreased 5hmC in cancer were lymphoid-related (FIG. 5). Likewise, 10 of the top 15 GSEA immune pathways (C7) identified in cancer over controls were pathways upregulated in monocytes, neutrophils/granulocytes, or myeloid cells (FIG. 30). Consistent with the GSEA results, buffy coat immunophenotyping identified a significant relative increase in granulocyte percentage and proportional relative decrease in lymphocyte percentage in cancer patients compared to non-cancer individuals (p-value<0.001), in a stage-dependent manner, while the percentage of monocytes was not significantly altered (FIG. 6). These results suggest that 5hmC profiling can capture changes that are induced in gDNA buffy coat of cancer patients.

### (ii) Cancer Classification Using Buffy Coat 5hmC Profiles

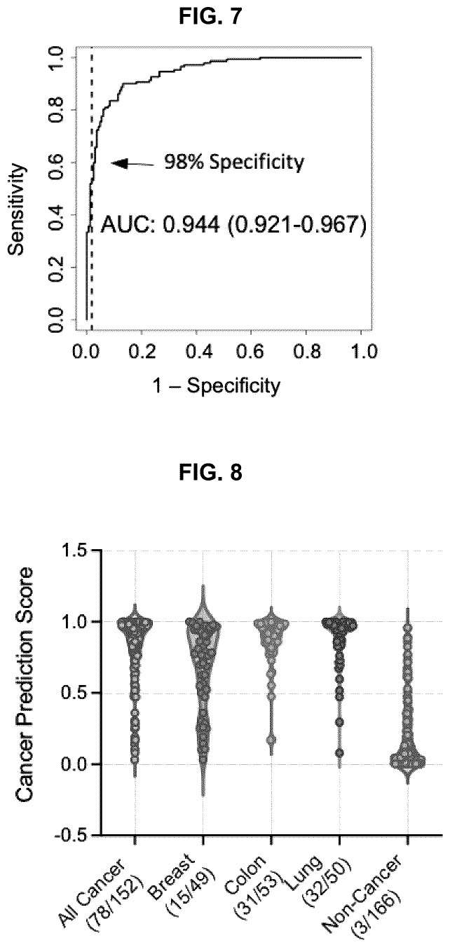

5hmC profiles obtained from buffy coat were used to build predictive models for cancer classification (FIG. 7). A binomial logistic regression prediction model was built using the elastic net regularization method (Friedman et al. (2010) J. Stat. Softw. 33: 1-22), using normalized 5hmC counts over genes. The model was calculated on 5 repetitions of 20-fold cross-validation, where a random 95% of the data was used for training and 5% was left out and used for test in each round of cross-validation. The training set yielded an out-of-fold performance of area under the ROC curve (auROC) of 0.944 (FIG. 3E), with 51.31% overall sensitivity (confidence interval [CI] 43.08%-59.49%) at 98% training specificity. This model is referred to herein as the “buffy coat (BC) model.”

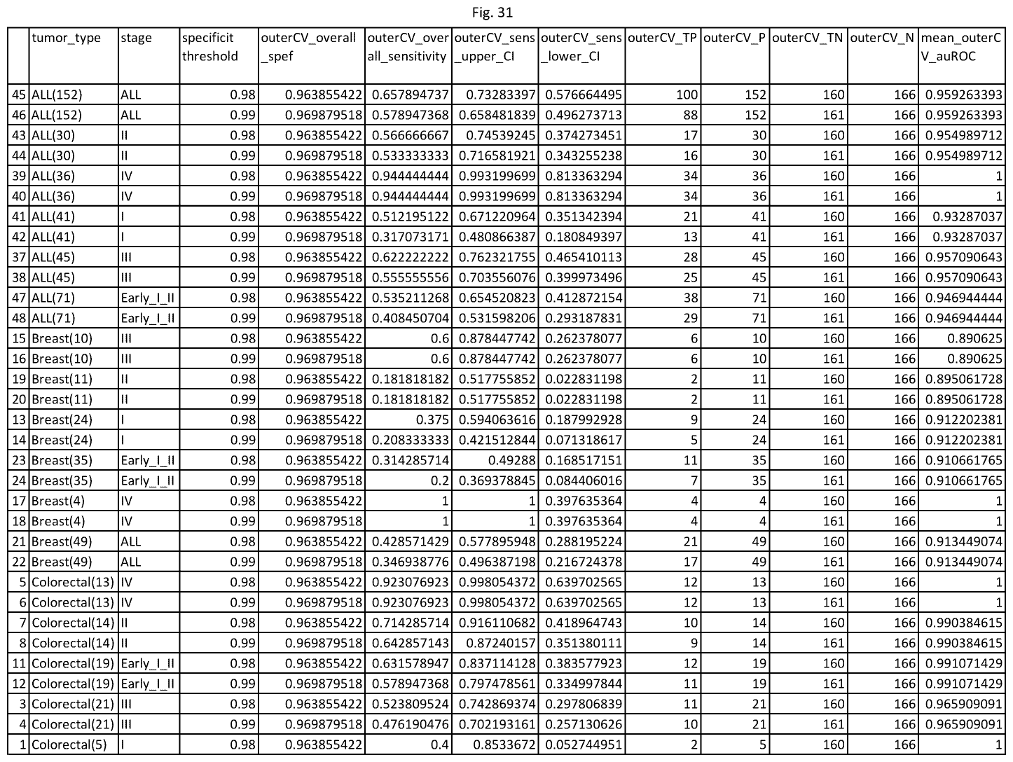

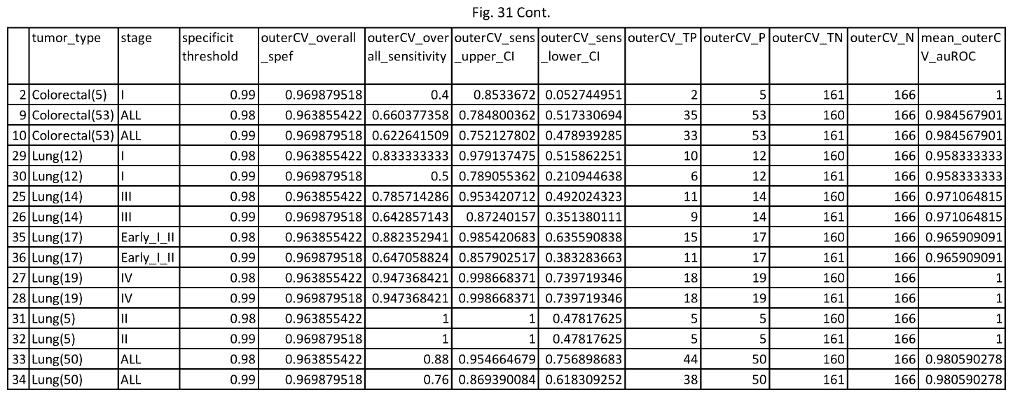

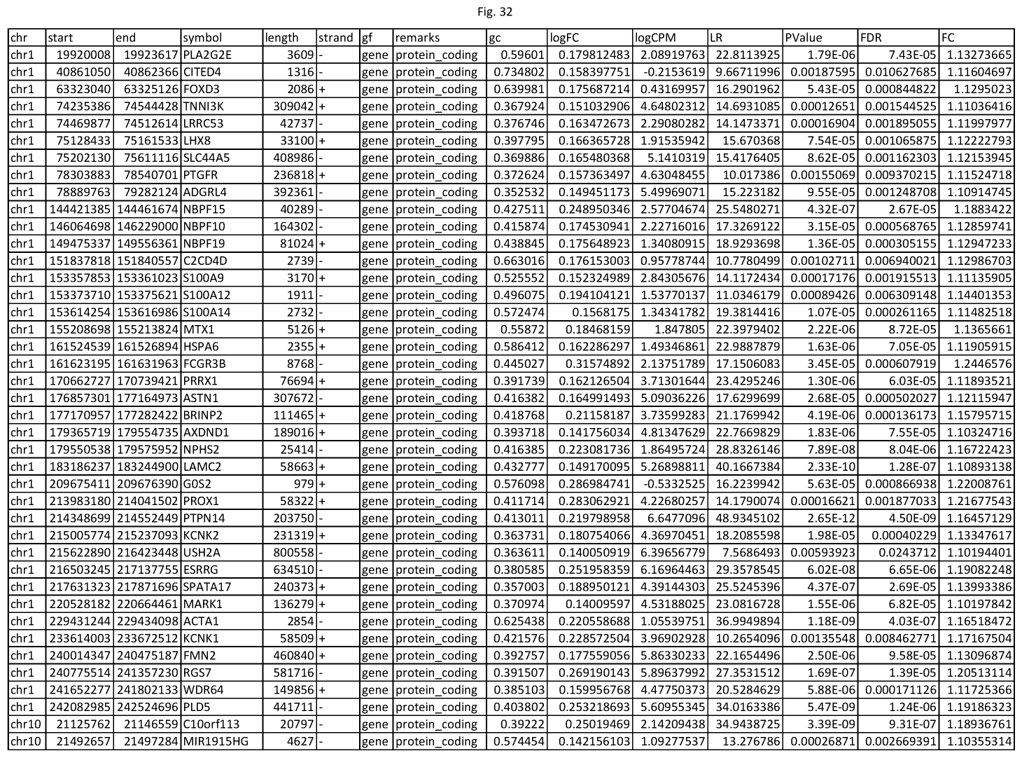

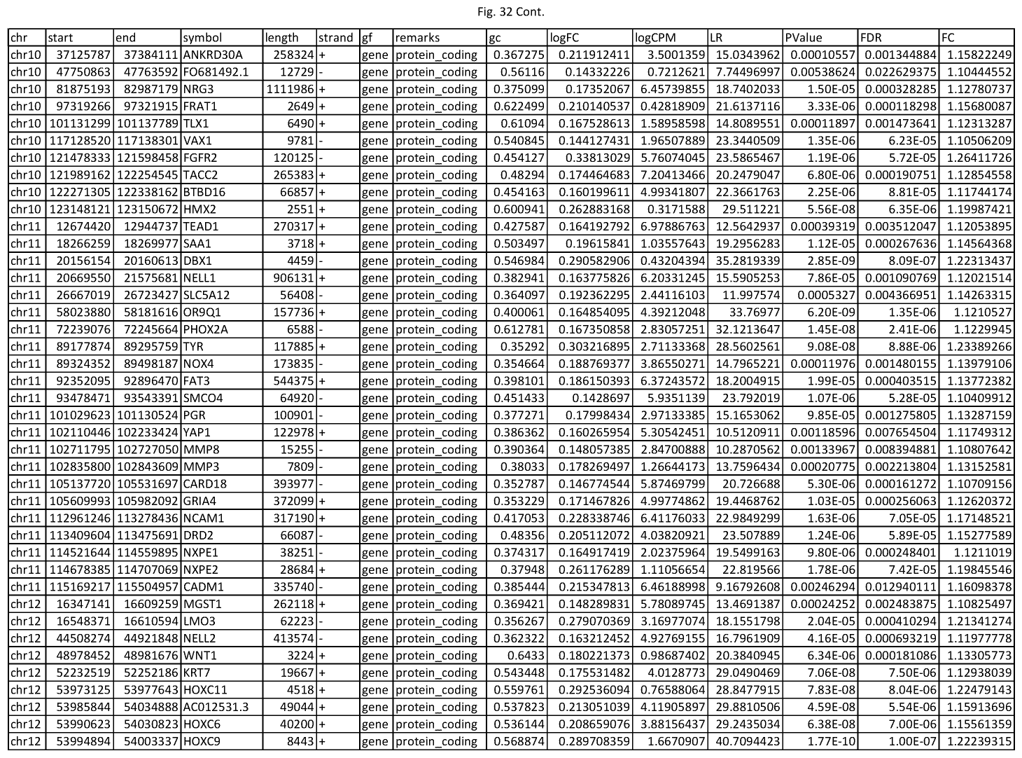

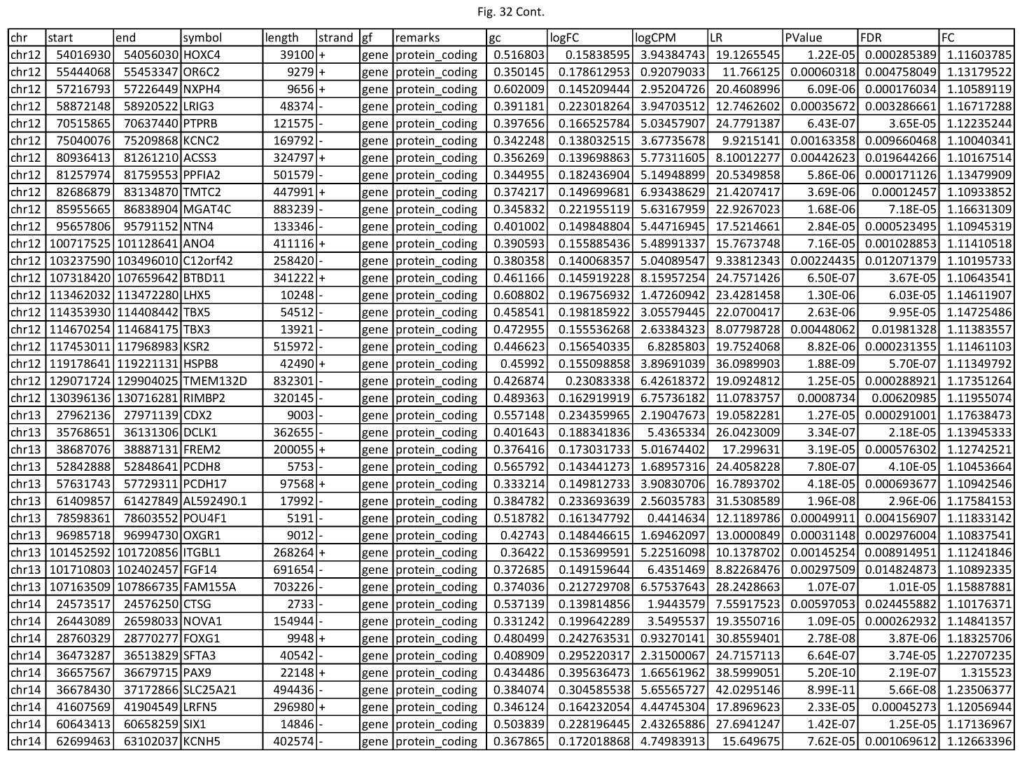

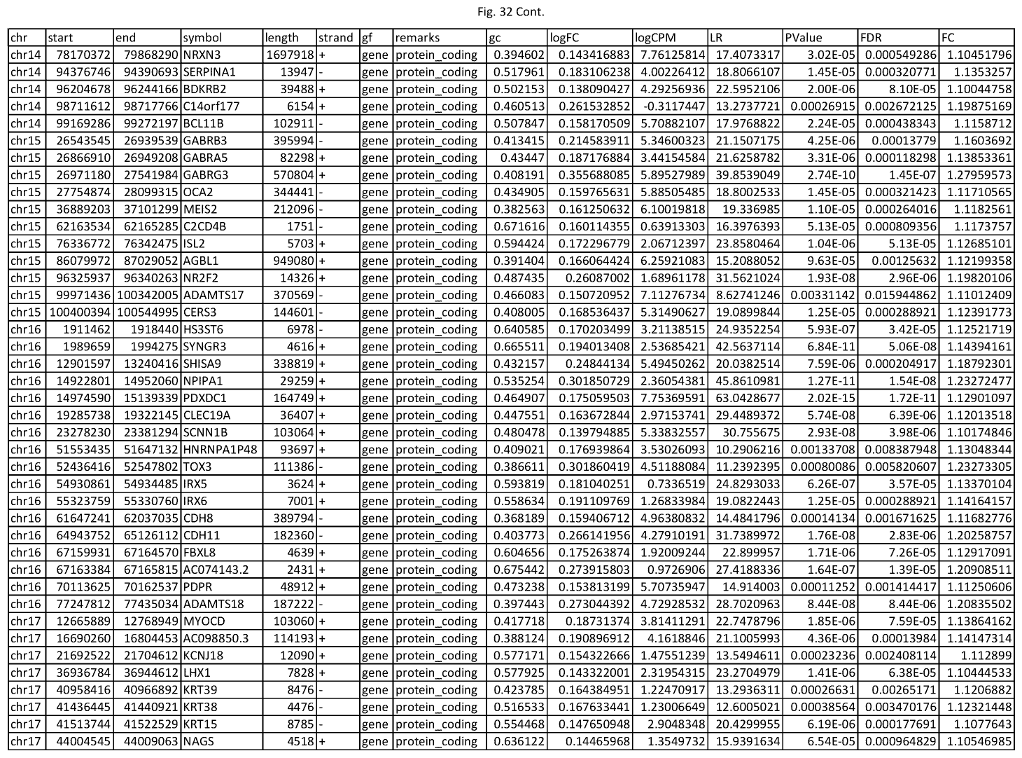

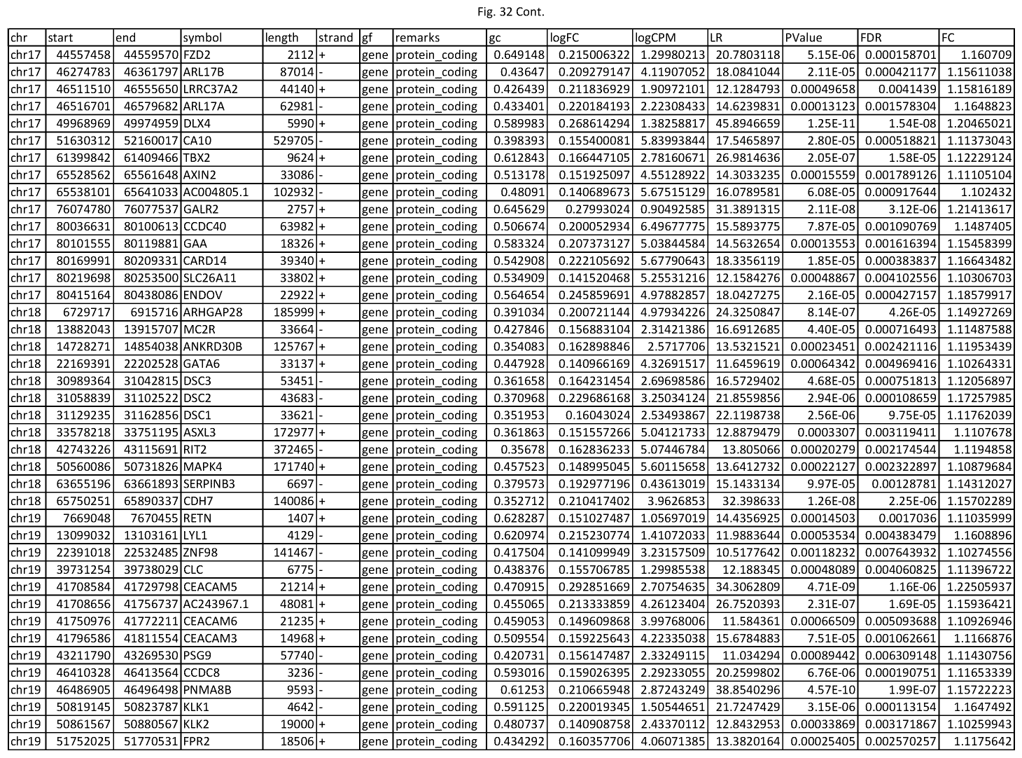

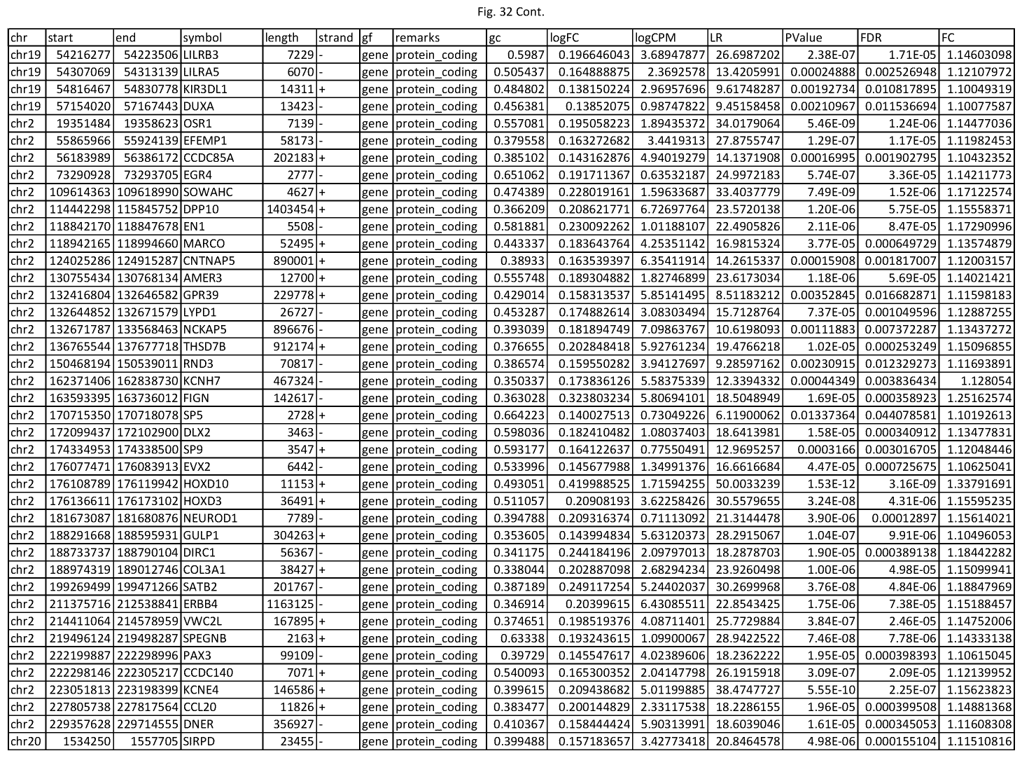

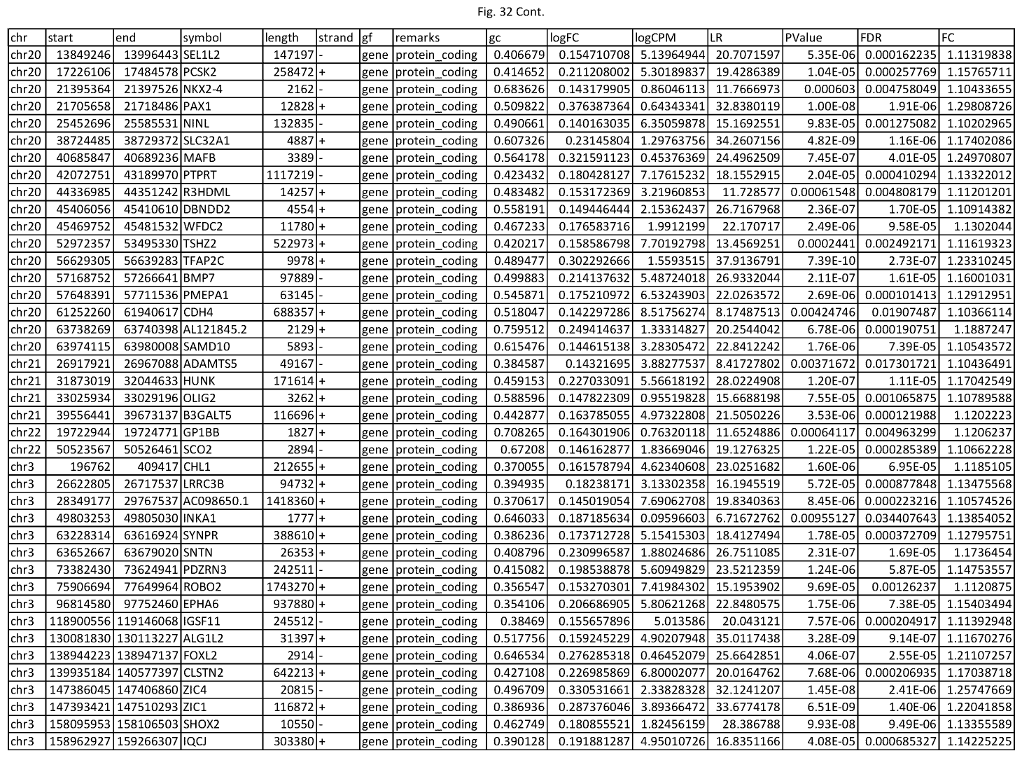

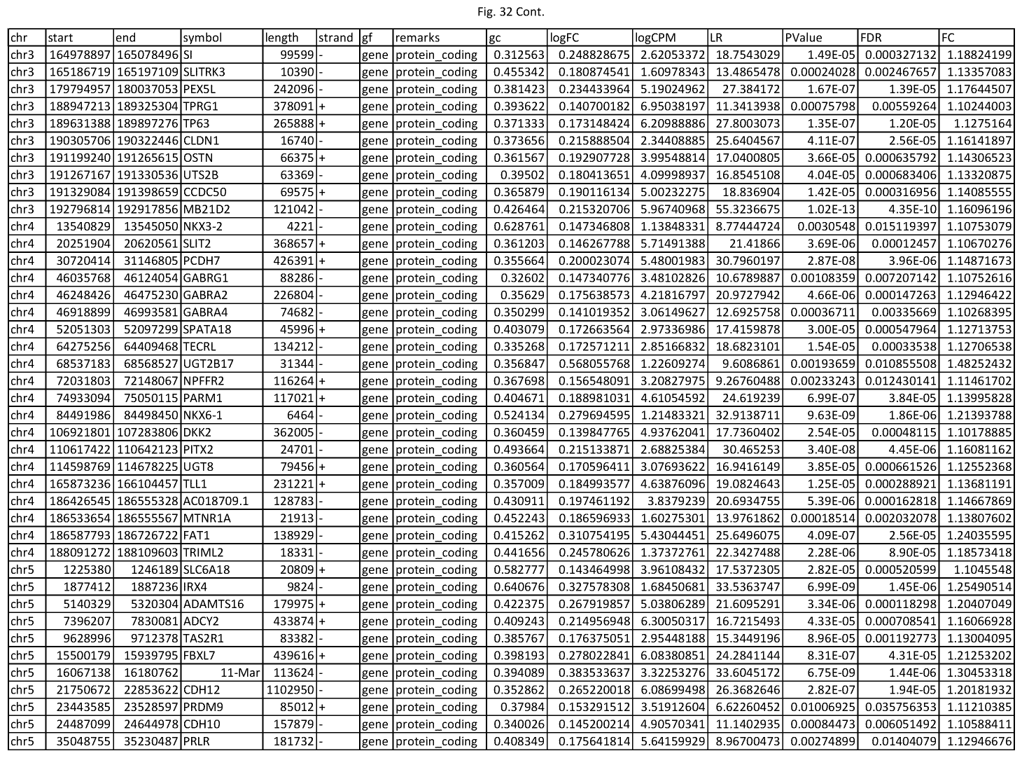

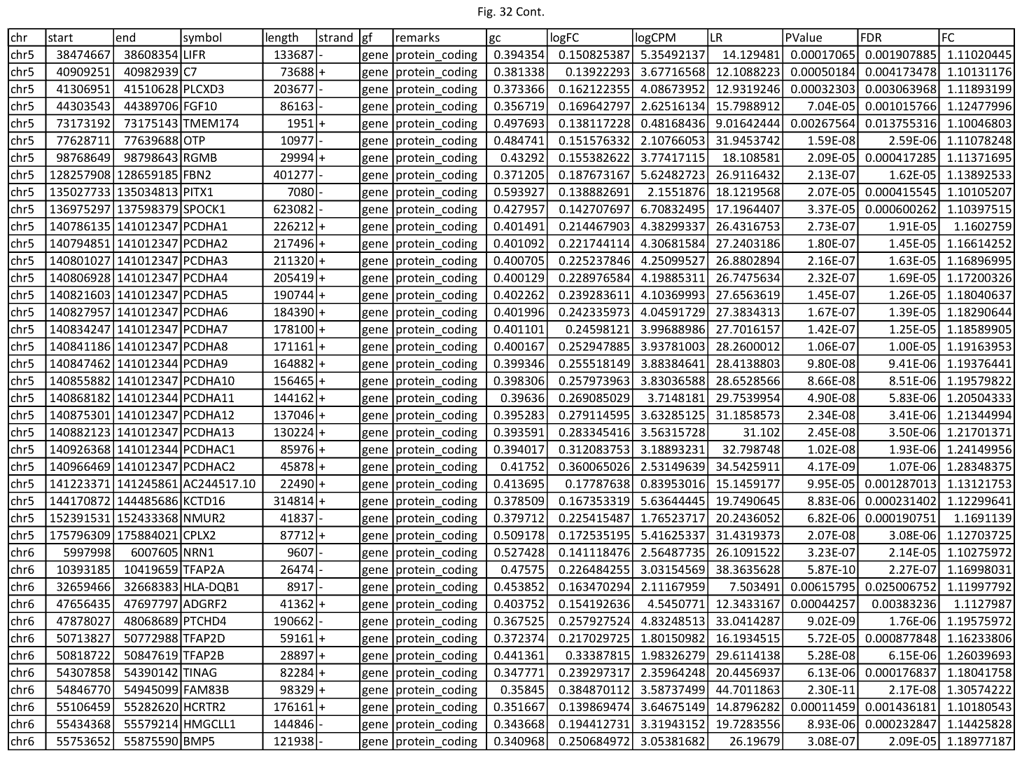

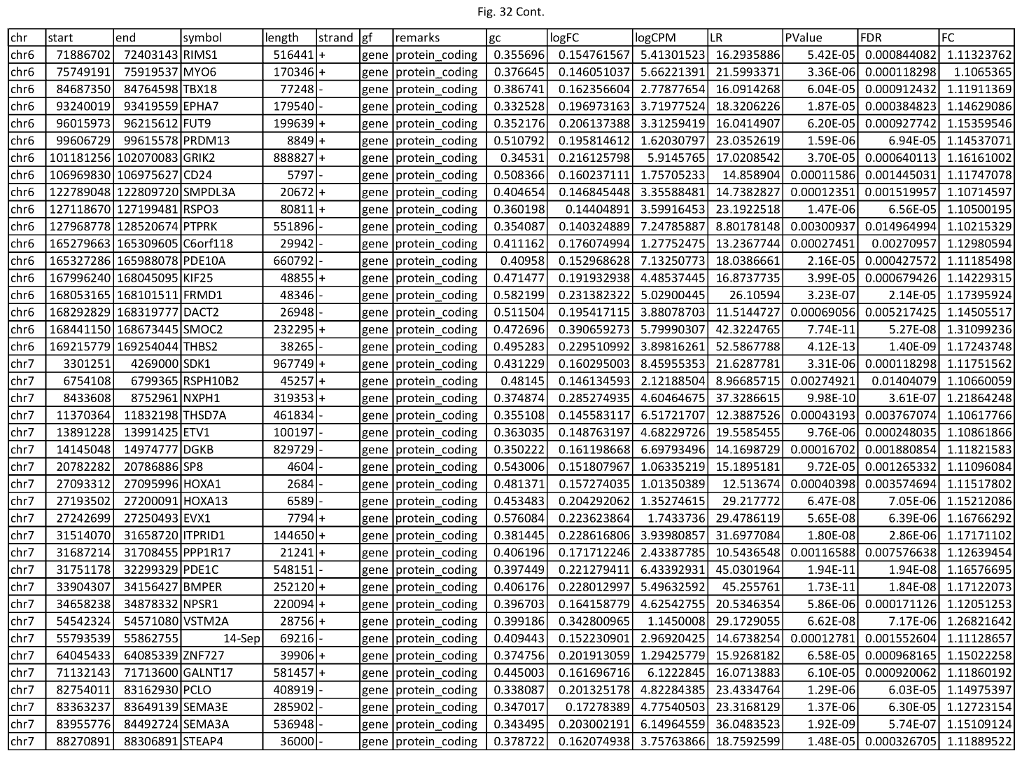

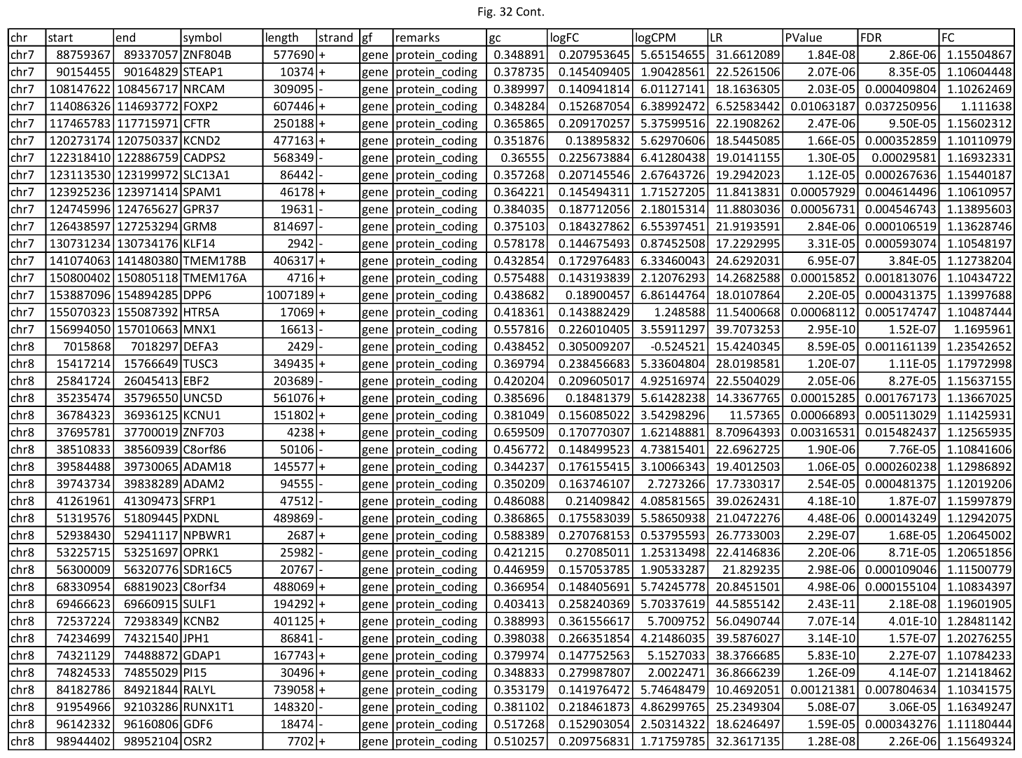

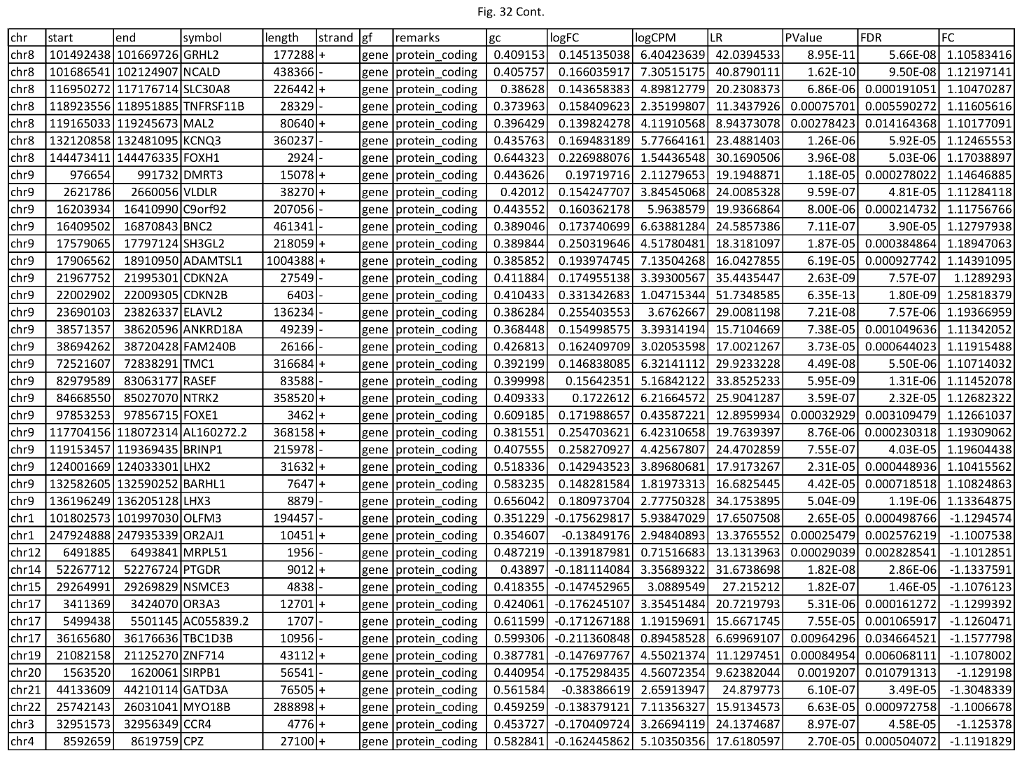

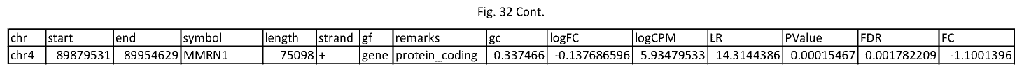

Investigation of cancer prediction scores based on cancer type revealed that samples from all three cancer types scored significantly higher than non-cancer controls (FIG. 8). Indeed, sensitivity observed for each cancer type were 30.6% for breast, 58.49% for colorectal and 64% for lung cancer at 98% training specificity threshold (FIG. 31). Together, these results show that buffy coat gDNA carries 5hmC signals that enable distinguishing between cancer and non-cancer samples.

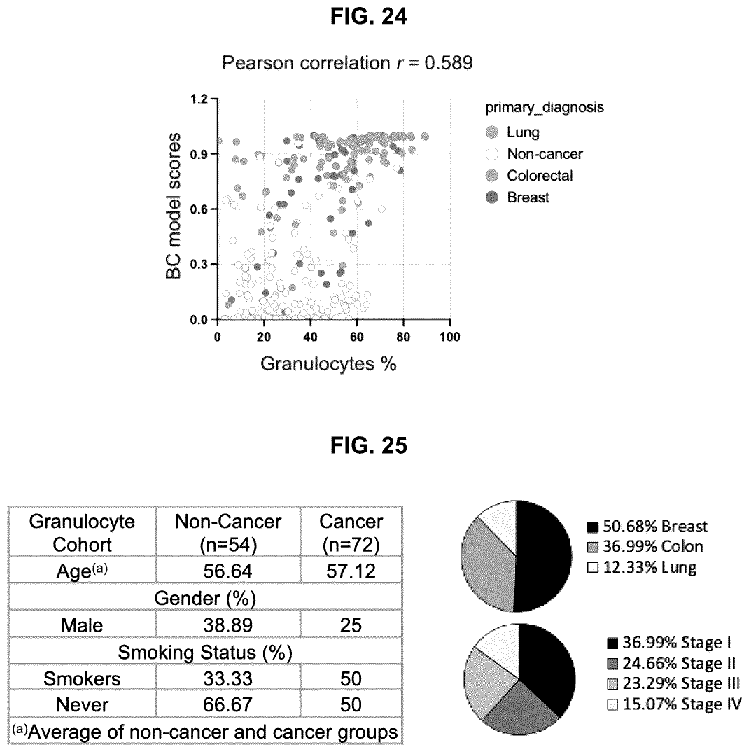

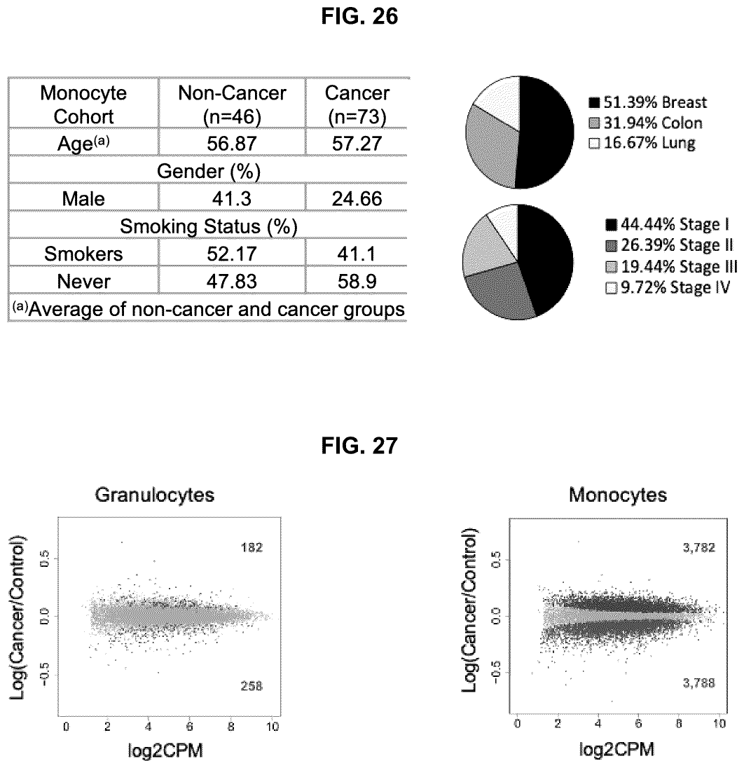

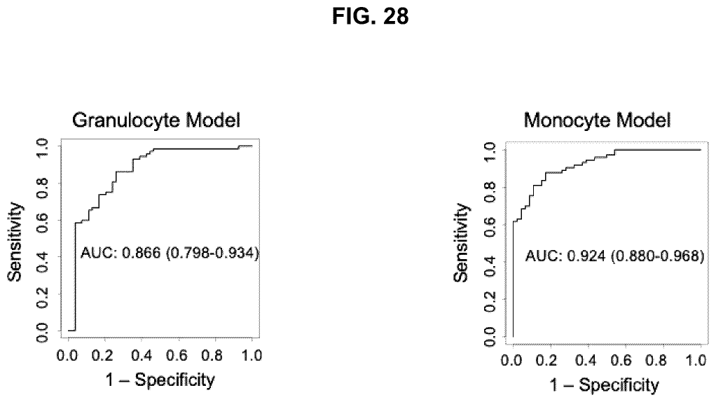

To investigate the biological basis for cancer classification using BC 5hmC profiles, we first examined the correlation between the cancer prediction scores and BC granulocyte percentage per sample as determined by immunophenotyping and determined that there is no significant correlation (FIG. 24). Next, we examined the 5hmC profiles of specific immune cell population(s) for cancer classification signal by enriching and analyzing the main lymphoid (B cells, CD4 and CD8 T cells and natural killer, or “NK,” cells) and myeloid (granulocytes and monocytes) populations present in the buffy coat. Consistent with the results obtained from GSEA (FIG. 6), myeloid populations isolated from buffy coat contained hundreds to thousands of DhMGs (FIG. 27) and yielded cancer classification models with outer-CV performance of AUC at 0.866 for granulocytes and 0.924 for monocytes (FIG. 28). These results demonstrate that peripheral myeloid blood cells of cancer patients have altered 5hmC profiles in comparison with non-cancer individuals. These contrasting 5hmC profiles can be utilized for detecting cancer and distinguishing between cancer and non-cancer samples, in addition to or in lieu of using the entire buffy coat hydroxymethylome.

(iii) Detection of Cancer-Specific 5hmC Signal and Building of Predictive Models for Cancer Detection Using cfDNA from Plasma:

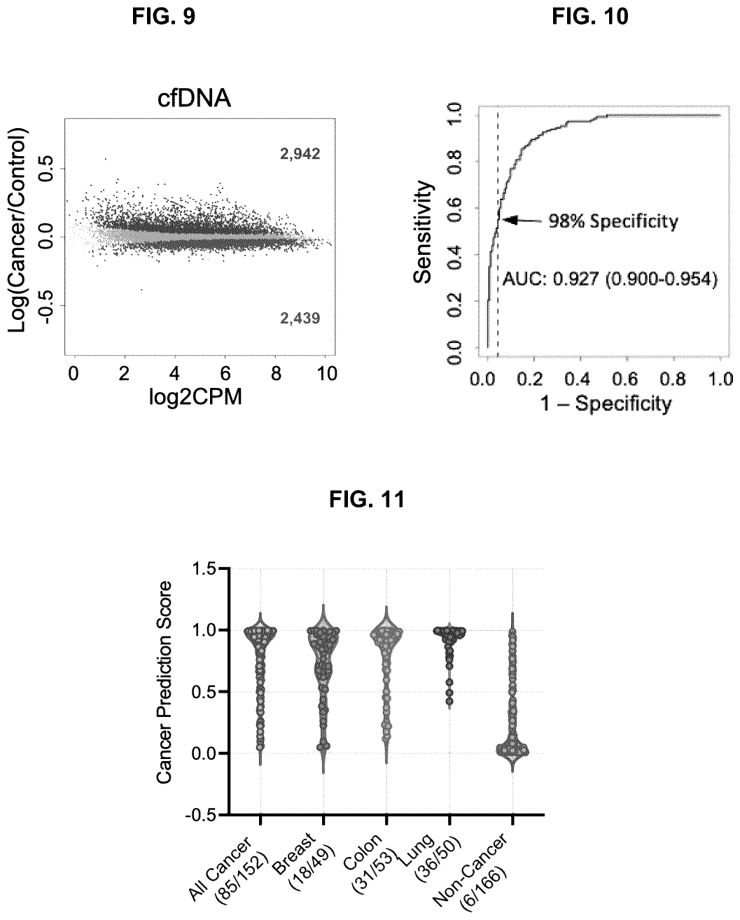

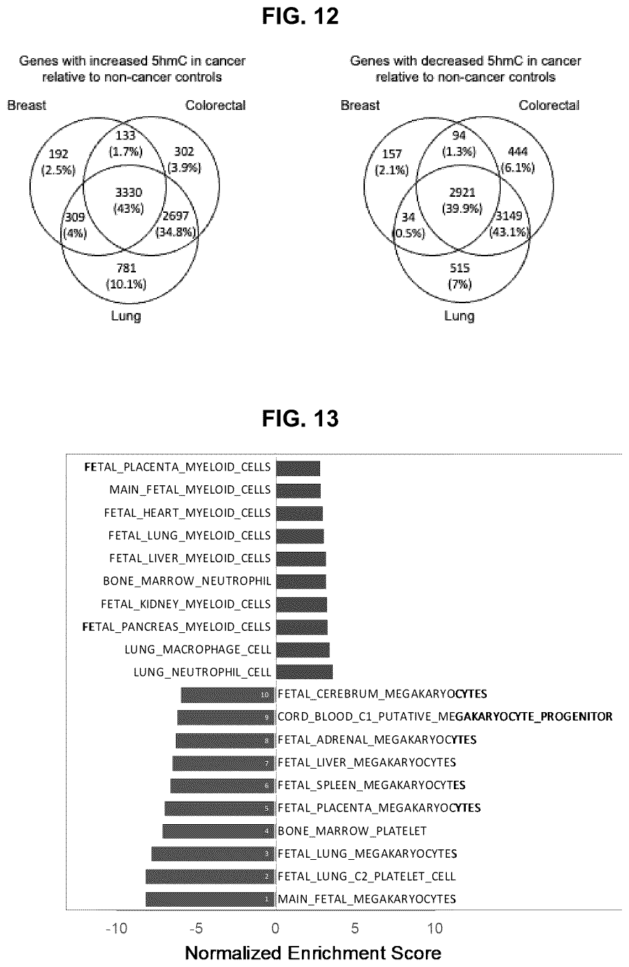

To understand how the 5hmC profiles of buffy coat gDNA compare with cfDNA 5hmC profiles, we sequenced the matched cfDNA samples from the same individuals and compared cancer samples with non-cancer controls (FIGS. 3-8). Relative to the comparison done using buffy coat profiles, cfDNA resulted in fewer but also substantial DhMGs, specifically 2,942 hyper-hydroxymethylated genes and 2,439 hypo-hydroxymethylated genes in cancer samples relative to non-cancer controls (FIGS. 9 and 30). Interestingly, there were more DhMGs for colorectal and lung cancers, with 5,614 and 13,746 DhMGs respectively, while breast had only 158 DhMGs (Table 2), consistent with previous reports that showed lower ctDNA levels in breast cancers, particularly at early stage (also see FIG. 12):

TABLE 2

| Cancer Type | Hyper-DhMG | Hypo-DhMG |
|---|---|---|
| All cancers | 2942 | 2439 |
| Breast | 110 | 48 |
| Colon | 2941 | 2673 |
| Lung | 7122 | 6624 |

Cancer prediction models built using 5hmC profiles along with WGS features resulted in an out-of-fold AUC of 0.927 (FIG. 10), with 52.63% overall sensitivity (CI 44.38%-60.78%) at 98% training specificity, similar to the performance observed for the buffy coat model. Prediction scores produced by the cfDNA model for each individual cancer type were significantly higher than for the non-cancer cohort (FIG. 11). Sensitivities observed for individual cancer types at 98% specificity were 36.73% for breast, 58.49% for colorectal and 72% for lung cancer (FIG. 31). Our results demonstrate that 5hmC features present on plasma cfDNA of solid tumors enable detection of breast, colorectal and lung cancers.

(iv) Comparing the 5hmC Gene Body Signals from Buffy Coat and Matched cfDNA:

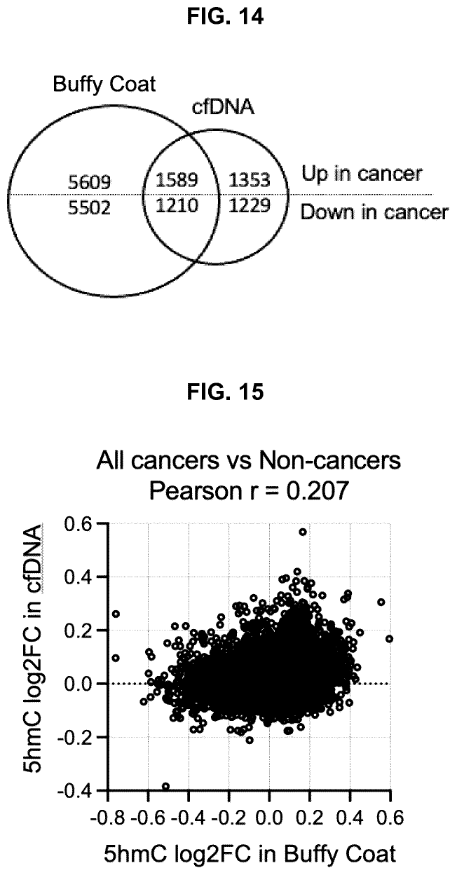

We next investigated the overlap between cancer induced changes identified using cfDNA 5hmC profiles to the ones identified using buffy coat gDNA 5hmC profiles by comparing cancers to non-cancer controls. Of the 13,910 DhMGs detected in the buffy coat and the 5,381 DhMGs identified in the matched cfDNA, i.e., cfDNA from the same patient, there were 2,799 DhMGs that were common (FIG. 14). This overlap was not statistically significant (p-value=1), indicating that the cfDNA and buffy coat DhMGs are different. Additionally, comparing the 5hmC fold change calculated for each gene by comparing cancers to non-cancer controls revealed a weak positive correlation coefficient of 0.21 between the cfDNA and buffy coat datasets (FIG. 15). These data altogether showed that buffy coat and matched cfDNA of cancer patients carry complementary and non-overlapping 5hmC modifications.

As one of the main challenges in liquid biopsy analyses is the detection of cancer at an early stage, we performed the same differential analysis using only early-stage samples. Strikingly, 5hmC analysis of gDNA isolated from the whole buffy coat of early-stage cancer samples compared to non-cancer samples identified 6,155 hyper- and 5,583 hypo-hydroxymethylated genes (FDR≤0.05), compared to only 61 hyper- and 34 hypo-hydroxymethylated genes using cfDNA 5hmC profiles (FIGS. 14 and 15). This result shows that the hydroxymethylome of buffy coat gDNA is already altered in early-stage cancer, when ctDNA levels are still low relative to late stage. Consistent with the changes in the BC hydroxymethylome in early-stage disease, a significant increase in granulocyte percentage was observed in early-stage buffy coat samples compared to non-cancers (FIG. 5). Altogether, these results suggest that the 5hmC signals from the buffy coat have potential to enable cancer detection even in early stages of the disease.

(v) Combining Buffy Coat and Matched cfDNA Models Improves Cancer Detection Performance:

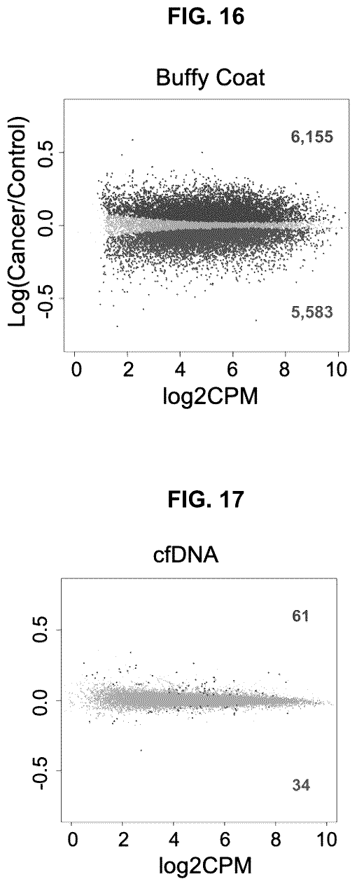

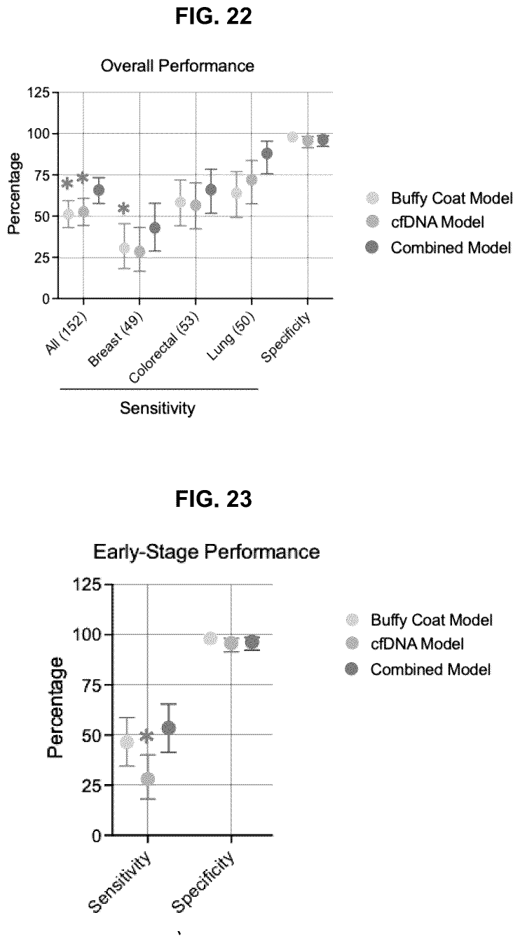

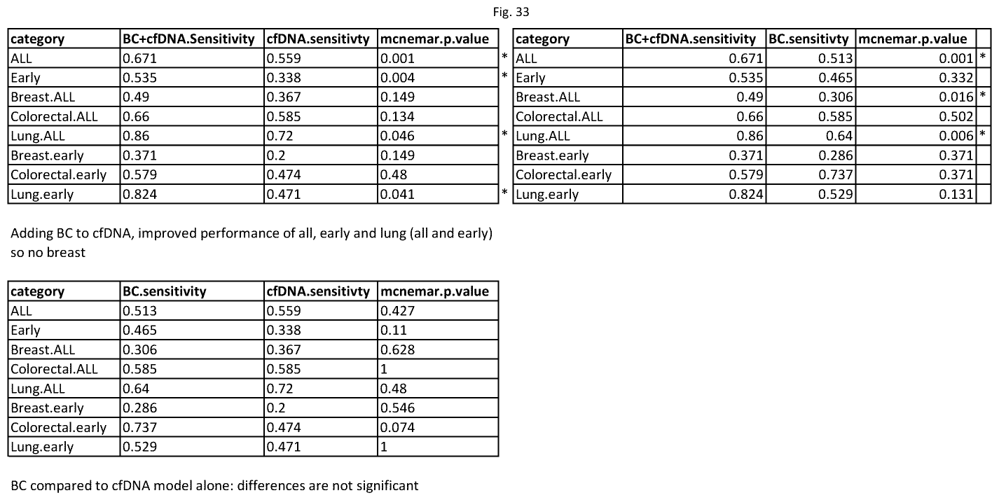

Given the findings that the buffy coat and the matched cfDNA models carry different and complementary signals, we next assessed whether combining these two models could increase cancer detection performance relative to using cfDNA or buffy coat models individually (FIGS. 16-21). Models built with feature sets from both buffy coat and the matched cfDNA performed with an AUC of 0.957 and overall sensitivity of 65.79% (CI 57.67%-73.28%) at 98% training specificity (FIGS. 16 and 29). FIG. 19 shows the cancer prediction score distribution for all samples in the study scored with the combined model. The number of true positive (TP) samples was significantly higher using the combined model compared to the individual models (FIG. 20), while the number of false positives (non-cancer samples scored as cancer by the model) was roughly identical among the three models (FIGS. 21 and 33). Notably, the overall sensitivity of the combined model at 98% training specificity was superior to the individual BC (p=0.001) and cfDNA (p=0.00033) models (FIG. 22). Furthermore, at 98% training specificity, the combined model discriminated early-stage cancer with superior performance (sensitivity 53.52% [CI 41.29%-65.45%]) compared to the compared to the cfDNA (sensitivity 28.17% [CI 18.13%-40.1%]) model (p=0.00014) (FIGS. 23 and 33). In conclusion, our results show that 5hmC signals from buffy coat can improve cancer detection provided by cfDNA models, especially for early-stage cancer detection.

### Summary of Experimental Work and Analysis:

The experimental work of this example indicates that the use of buffy coat 5hmC analysis, particularly when combined with cfDNA 5hmC analysis, lends itself to an improved method for detecting cancer and for distinguishing cancer from non-cancer blood samples. Using the liquid biopsy technique, we sequenced the 5hmC-containing fragments of the whole buffy coat gDNA, which is available in the same phlebotomy sample collected to extract the cfDNA from the plasma fraction. Instead of accessing the whole buffy coat, most of the research to date has involved sequencing only fractions of the buffy coat, such as PBMCs (peripheral blood mononuclear cells; see Zhang et al. (2018) Clin. Epigenetics 10: 8), which exclude the granulocytes; or isolated T and B lymphocytes, with a few profiling the different immune populations found in the buffy coat (Parashar (2018) Bmc Cancer 18: 574; Wernig-Zorc et al. (2019) Epigenetic Chromatin 12: 4; Koestler et al. (2012) Cancer Epidem. Prev. Biomarkers 21: 1293-1302; Manoochehri et al. (2021) doi: 10:10.21203/rs.3.rs-508197/v2). Some other reports have sequenced paired cfDNA-white blood cells, to distinguish CHIP (clonal hematopoiesis of indeterminate potential) from their ctDNA-derived counterparts (Chan et al. (2020) Cancers 12:2277; Song et al. (2017), cited supra) and not to identify specific signals coming from the buffy coat, as done here. The present strategy was to combine 5hmC features from the plasma cfDNA with 5hmC signals derived from the whole buffy coat gDNA, to build cancer prediction models.

We observed a relative increase in granulocytes in the buffy coat of cancer samples compared to the controls (FIG. 5), with late-stage cancer samples having the highest percentage of granulocytes. In parallel, Allen et al. (2020) reported in a murine model of breast tumor that neutrophils, the main population of granulocytes in the blood, were the immune cell type increasing in peripheral blood during cancer progression (Allen et al. (2020) Nat. Med. 26: 1125-34). Likewise, Engblom et al. (2017) also identified a subpopulation of neutrophils increased by the presence of lung cancers (Engblom et al. (2017) Science 358:6367). These new results support the literature reporting a tumor-driven skew in hematopoiesis to myeloid populations, although the increase in granulocyte percentage may be primarily indicative of inflammation.

We next identified significant differences in the 5hmC profile of the peripheral buffy coat gDNA between cancer samples and non-cancer control samples, which in turn enabled the building of a predictive multi-cancer detection model (FIG. 7) with AUC of 0.944, for a cohort comprised of 46.4% early-stage cancers, the most difficult cancer stages to be detected. Unlike the lymphoid populations isolated from the buffy coat, both granulocytes and monocytes yielded cancer predictive models with an AUC of 0.866 and 0.924, respectively (FIG. 28), demonstrating that these myeloid cells, at least in part, contribute to the cancer signal derived from the buffy coat gDNA. Granulocytes and monocytes have the same hematopoietic precursor, GMP (granulocyte-monocyte progenitor), which is increased by tumor-derived factors and able to differentiate into immature myeloid cells (Wu et al. (2014), cited previously). Several DhMGs enriched in the cancer cohort and associated with myeloid functions (FIGS. 4, 6 and 29) resemble MDSCs, an immature population of myeloid cells that suppress immune functions and support tumor progression. Arginase (ARG1), elastase (ELANE), the cytokine G-CSF (CSF3) (Casbon et al. (2015), cited supra), S100A8, S100A9 and the metallopeptidase MMP8 (Ouzounova et al. (2017) Nat. Commun. 8: 14979) were all previously implicated in the development and function of MDSCs. Taken together, the results are consistent with the emerging role of the myeloid arm of the peripheral immune system being altered in cancer and demonstrate alterations in the hydroxymethylome of buffy coat gDNA, which are valuable for cancer detection.

Next, a cancer prediction model was built using 5hmC and WGS features from the plasma cfDNA of matching patients. The performance of the cfDNA model was similar to that of the buffy coat model, notwithstanding that the DhMGs identified in the buffy coat and cfDNA datasets were different (FIGS. 14 and 15). Notably, the colorectal and lung cancer samples exhibited thousands of DhMGs using either the buffy coat gDNA or the cfDNA features. For breast and early-stage cancers, buffy coat gDNA yielded thousands of DhMGs (FIGS. 16 and 17 and Table 1), while cfDNA had only 95 DhMGs (FIG. 15). These findings indicate that the DhMGs from buffy coat gDNA and cfDNA complement each other in a method for improving cancer detection.

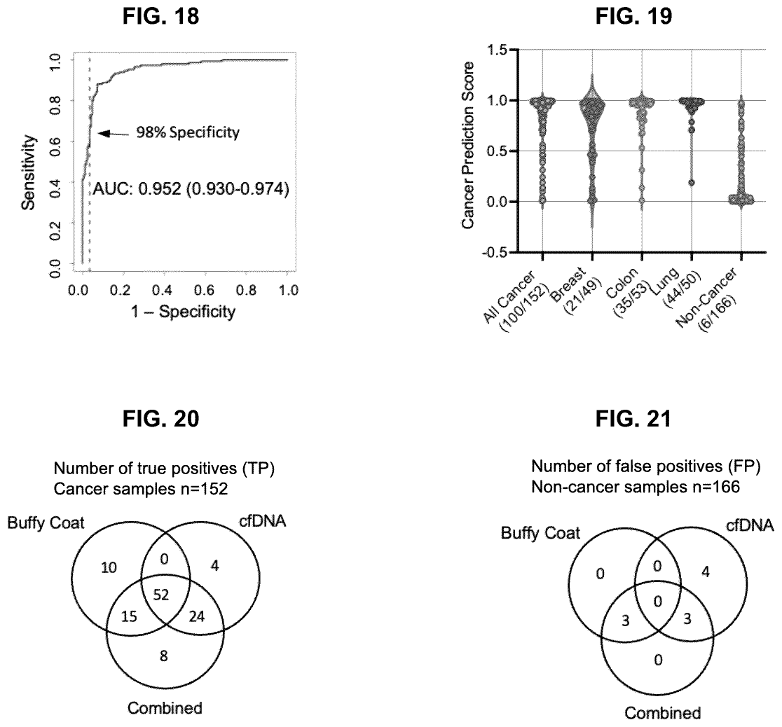

The approach of combining the 5hmC feature sets from buffy coat and the matched cfDNA yielded a cancer prediction model with superior performance relative to the individual models, with an AUC of 0.952 and overall sensitivity of 65.79 at 98% training specificity, compared to 51.31% for BC and 52.63% for the cfDNA models (FIGS. 18-23 and 31). Remarkably, the early-stage sensitivity of the combined model (53.52%) improved the early-stage sensitivity of the cfDNA model (28.17%) at 98% training specificity (FIGS. 23 and 33). As a conclusion, our data demonstrated that 5hmC profiles derived from the BC improve cancer prediction performance achieved by cfDNA models, in particular for early-stages of the disease, where is the main limitation of cfDNA-based liquid biopsy assays.

Comparison of the differential features in buffy coat and cfDNA 5hmC profiles revealed non-overlapping feature sets that can be utilized for cancer classification. Combining these two models resulted in an enhanced combination model with superior classification power with regard to the detection of cancer in early stages. To the best of our knowledge, the work described in this Example is the first time that 5hmC from the whole buffy coat layer in solid tumors has been sequenced, showing epigenetic reprogramming of buffy coat gDNA in cancer and the potential to be applied in liquid biopsy assays to improve cancer diagnostics, particularly for early-stage detection.

1. A method for analyzing buffy coat in a peripheral blood sample obtained from a patient, wherein the method comprises:
obtaining a buffy coat hydroxymethylation signature for the patient by extracting genomic DNA (gDNA) from the buffy coat without separating the buffy coat into individual cell types;
sequencing the gDNA in a manner that identifies 5-hydroxymethylcytosine (5hmC)-containing sites therein; and
determining the extent of hydroxymethylation of the sequenced gDNA at each of a plurality of hydroxymethylation biomarker loci in a reference data set for a population group of individuals who have at least one shared characteristic, wherein the biomarker loci are preselected as differentially hydroxymethylated with respect to the at least one shared characteristic; and
reporting the extent of hydroxymethylation at each locus as the buffy coat hydroxymethylation signature.

2. The method of claim 1, wherein the at least one shared characteristic comprises having cancer.

3. The method of claim 1, wherein the at least one shared characteristic comprises having a particular type of cancer.

4. The method of claim 1, wherein the at least one shared characteristic comprises not having cancer.

5. The method of claim 1, wherein the at least one shared characteristic comprises not having a particular type of cancer.

6. The method of claim 1, wherein each hydroxymethylation biomarker locus is selected as exhibiting differential hydroxymethylation in a manner that correlates with having cancer or a particular type of cancer.

7. The method of claim 6, wherein differential hydroxymethylation is determined using a p-value of less than or equal to 0.05 using a linear regression F-test.

8. The method of claim 1, further comprising generating a buffy coat gDNA-based probability score that the patient has cancer based on the buffy coat hydroxymethylation signature.

9. The method of claim 8, further comprising combining the buffy coat gDNA-based probability score with an additional feature value for at least one additional feature type to characterize the likelihood that the patient has cancer.

10. A method for analyzing a peripheral blood sample obtained from a patient, wherein the method comprises:
(a) obtaining a buffy coat hydroxymethylation signature for the patient by
(i) extracting genomic DNA (gDNA) from the buffy coat;
(ii) sequencing the gDNA in a manner that identifies 5-hydroxymethylcytosine (5hmC)-containing sites therein;
(iii) determining the extent of hydroxymethylation of the sequenced gDNA at each of a plurality of hydroxymethylation biomarker loci in a reference data set for a population group of individuals who have at least one shared characteristic, wherein the biomarker loci are preselected as differentially hydroxymethylated with respect to the at least one shared characteristic, thereby providing the buffy coat hydroxymethylation signature as the extent of hydroxymethylation at each locus;
(b) obtaining a cfDNA hydroxymethylation signature for the patient by
(i) isolating cfDNA from plasma in a peripheral blood sample obtained from the patient;
(ii) enriching for hydroxymethylated DNA in the cfDNA, amplifying the hydroxymethylated DNA, sequencing the amplified hydroxymethylated DNA in a manner that identifies 5-hydroxymethylcytosine (5hmC)-containing fragments in the DNA, and determining the extent of hydroxymethylation of the sequenced cfDNA at each of a plurality of cfDNA hydroxymethylation biomarker loci preselected as differentially hydroxymethylated with respect to the at least one shared characteristic, thereby providing the cfDNA hydroxymethylation signature as the extent of hydroxymethylation at each locus; and
(c) combining the buffy coat gDNA hydroxymethylation signature and the cfDNA hydroxymethylation signature in a manner that provides a classification model suitable for generating a composite probability score representing the likelihood that the patient has cancer or a particular type of cancer.

11. The method of claim 10, wherein the composite probability score represents the likelihood that the patient has cancer.

12. The method of claim 10, wherein the composite probability score represents the likelihood that the patient has a particular type of cancer.

13. The method of claim 12, wherein the particular type of cancer is breast cancer.

14. The method of claim 12, wherein the particular type of cancer is colorectal cancer.

15. The method of claim 12, wherein the particular type of cancer is lung cancer.

16. The method of claim 10, further including combining the composite probability score with an additional feature value for at least one additional feature type to characterize the likelihood that the patient has cancer.

17. The method of claim 16, wherein the additional feature type comprises DNA fragment size distribution, copy number variation, cfDNA concentration, methylation profile, T-cell-inflamed gene expression profile, circulating tumor DNA count, serum CA19-9 level, serum CA125 level, IDO-1 expression, T-cell count, T-cell percentage, inflammation gene signature, myeloid-derived suppressor cell count, lymphocyte count, deficient mismatch repair, tumor mutational burden, presence or absence of germline mutations, a patient-specific clinical parameter, and combinations of any of the foregoing.

18. The method of claim 17, wherein the additional feature type comprises:
the number of cfDNA fragments in each of at least two nonoverlapping size ranges;
copy number variation in the cfDNA sample;
concentration of cfDNA in the cfDNA sample;
a patient-specific clinical parameter; and
combinations of any of the foregoing.

19. The method of claim 17, wherein the patient-specific clinical parameter is selected from lesion size; lesion grade; lesion stage; lesion location; patient age; patient weight; patient gender; patient ethnicity; cigarette smoking status; and exposure or lack of exposure to a known carcinogen.

20. The method of claim 16, wherein the combining comprises a stacked ensemble analysis.

21. The method of claim 17, wherein the combining comprises a stacked ensemble analysis.

22. The method of claim 18, wherein the combining comprises a stacked ensemble analysis.

23. The method of claim 20, wherein the buffy coat hydroxymethylation signature is used as a base model in the stacked ensemble analysis.

24. A method for analyzing a blood sample obtained from a patient, wherein the method comprises:
(a) isolating a specific cell type from the buffy coat layer of a peripheral blood sample obtained from the patient;
(b) obtaining a buffy coat cell-specific hydroxymethylation signature for the patient by (i) extracting genomic DNA (gDNA) from the isolated specific cell type;
(ii) sequencing the gDNA in a manner that identifies 5-hydroxymethylcytosine (5hmC)-containing sites therein; and
(iii) determining the extent of hydroxymethylation of the sequenced gDNA at each of a plurality of hydroxymethylation biomarker loci in a reference data set for a population group of individuals who have at least one shared characteristic, wherein the biomarker loci are preselected as differentially hydroxymethylated with respect to the at least one shared characteristic, thereby providing the buffy coat cell-specific hydroxymethylation signature as the extent of hydroxymethylation at each locus;
(c) isolating cfDNA from plasma in a peripheral blood sample obtained from the same patient;
(d) obtaining a cfDNA hydroxymethylation signature for the patient by enriching for hydroxymethylated DNA in the cfDNA, amplifying the hydroxymethylated DNA, sequencing the amplified hydroxymethylated DNA in a manner that identifies 5-hydroxymethylcytosine (5hmC)-containing fragments in the DNA, and determining the extent of hydroxymethylation of the sequenced cfDNA at each of a plurality of cfDNA hydroxymethylation biomarker loci preselected as differentially hydroxymethylated with respect to the at least one shared characteristic, thereby providing the cfDNA hydroxymethylation signature as the extent of hydroxymethylation at each locus; and
(e) combining the buffy coat cell-specific hydroxymethylation signature and the cfDNA hydroxymethylation signature in a manner that provides a classification model suitable for generating a composite probability score representing the likelihood that the patient has cancer or a particular type of cancer.

25. The method of claim 24, wherein the specific cell type is a granulocyte.

26. A method for determining a likelihood that a patient has cancer, comprising determining a percentage of granulocytes in a buffy coat fraction extracted from a peripheral blood sample obtained from the patient and comparing the percentage to an established percentage of granulocytes in a reference standard, wherein an elevated percentage of granulocytes in the buffy coat fraction is indicative of a likelihood that the patient has cancer.
